(19) United States
(12) Patent Application Publication
(10) Pub. No.: US 2023/0049356 A1
(43) Pub. Date: Feb. 16, 2023

(54) SYSTEMS AND METHODS FOR PHARMACEUTICAL INJECTION SUPPLY MANAGEMENT

(71) Applicants: Samson Pollak, Austin, TX (US); Kevin Mayo, Bloomington, IN (US)

(72) Inventors: Samson Pollak, Austin, TX (US); Kevin Mayo, Bloomington, IN (US)

(21) Appl. No.: 17/402,176

(22) Filed: Aug. 13, 2021

(51) Int. Cl. G16H 20/17; G06F 9/451; G06F 17/18

(52) U.S. Cl. CPC G16H 20/17; G06F 9/451; G06F 17/18; G06Q 30/0283

(57) ABSTRACT

The present disclosure relates to systems and methods for pharmaceutical injection supply management, and in particular, recommending an optimal amount of pharmaceutical injection supply for a user to load into a dispenser. Implementations of the systems and methods discussed herein can provide, via a mobile application, a recommendation to a pharmaceutical user of the amount of substance to load into their substance pump during a site change. The recommendation can be optimized to minimize substance costs and substance waste, or to accommodate a target replacement date and time.

### FIELD OF THE DISCLOSURE

The present application relates generally to systems and methods for management and monitoring of pharmaceutical injection supplies, and more particularly, to methods and systems for pharmaceutical injection supply management.

### BACKGROUND

Management of pharmaceutical injection supplies can be difficult because users can either load too much of the pharmaceutical injection supply and risk having it expire, or load too little and risk not having enough to satisfy medical needs or having to perform frequent refills. Moreover, performing additional refills can be wasteful because any remaining pharmaceutical injection supply (e.g. part of a vial or supply unit) often needs to be thrown away during the refills.

### SUMMARY

The present disclosure is directed to recommending an optimal amount of pharmaceutical injection supply for the user to load into their dispenser. For example, the pharmaceutical injection supply can be insulin, and the dispenser can be an insulin pump. In particular, the present disclosure can provide, via a mobile application, a recommendation to a pharmaceutical user of the amount of substance to load into their substance pump during a site change. The recommendation can be optimized to minimize substance costs and substance waste, or to accommodate a target replacement date and time.

At least one aspect of this disclosure is directed to a system. The system can include a network receiver configured to receive dispensing activity of a pump communicatively coupled to the network receiver. The system can include one or more processors. The one or more processors can be configured to detect, via the network receiver, a plurality of dispensing events, wherein each of the plurality of dispensing events includes a timestamp and an amount of a substance that was dispensed, the substance having one or more predetermined refill amounts and an expiration time. The one or more processors can be configured to calculate, from the plurality of dispensing events, an amount of substance dispensed per dispensing event. The one or more processors can be configured to select, based on the amount of substance dispensed per dispensing event and the expiration time, a refill amount of the one or more predetermined refill amounts. The one or more processors can be configured to provide, via an interactive interface, an indication of the selected refill amount.

In some embodiments, the one or more processors can be configured to receive, from the interactive interface, a first expenditure per unit of the substance and a second expenditure per refill of the pump. The one or more processors can be configured to select the refill amount based on the amount of substance dispensed per dispensing event, the expiration time, the first expenditure, and the second expenditure.

In some embodiments, the one or more processors can be configured to calculate the refill amount as proportional to a mean and a standard deviation of the amount of the substance that was dispensed during the plurality of dispensing events. The one or more processors can be configured to identify an inverse of a probability density function of the plurality of dispensing events.

In some embodiments, the one or more processors can be configured to identify, based on an expected refill amount of the one or more predetermined refill amounts and the amount of substance dispensed per dispensing event, an expected amount of substance remaining in the pump at a first expected refill time of the substance based on the expected refill amount. The one or more processors can be configured to identify, based on the selected refill amount and the amount of substance dispensed per dispensing event, an estimated amount of substance remaining in the pump at a second expected refill time of the substance based on the selected refill amount. The one or more processors can be configured to calculate a difference between the expected amount of substance remaining and the estimated amount of substance remaining. The one or more processors can be configured to provide, via the interactive interface, the difference.

In some embodiments, the one or more processors can be configured to generate an expenditure comparison based on (i) the first expenditure and the difference, and (ii) the second expenditure and a ratio between a first refill frequency of the pump based on the expected amount of substance and a second refill frequency of the pump based on the estimated amount of substance. The one or more processors can be configured to provide, via the interactive interface, the expenditure comparison.

In some embodiments, the one or more processors can be configured to receive, via the interactive interface, a preferred refill time of the substance. The one or more processors can be configured to identify an estimated amount of substance remaining in the pump at the preferred refill time of the substance.

In some embodiments, the amount of substance dispensed per dispensing event comprises a basal rate and a bolus rate of substance dispensing.

In some embodiments, each of the one or more predetermined refill amounts of the substance corresponds to a size of a container containing the substance.

In some embodiments, the one or more processors can be configured to calculate a first metric by multiplying the first expenditure by the amount of substance dispensed per dispensing event. The one or more processors can be configured to calculate a second metric by adding the second expenditure to a product of the first expenditure and an estimated amount of substance remaining in the pump at the expiration time of the substance or an expected refill time of the substance, the estimated amount of substance remaining based on a current amount of the substance and the amount of substance dispensed per dispensing event. The one or more processors can be configured to identify the probability density function of the plurality of dispensing events based on a proportion between a first metric and a second metric.

In some embodiments, the one or more processors can be configured to generate a graph comprising the amount of the substance that was dispensed during each of the plurality of dispensing events. The one or more processors can be configured to calculate a line of best fit of the amount of the substance that was dispensed during each of the plurality of dispensing events. The one or more processors can be configured to display, via the interactive interface, the graph and the line of best fit.

At least one aspect of this disclosure is directed to a method. The method can include detecting, by a computing device, via a network receiver configured to receive dispensing activity of a pump communicatively coupled to the network receiver, a plurality of dispensing events, wherein each of the plurality of dispensing events includes a timestamp and an amount of a substance that was dispensed, the substance having one or more predetermined refill amounts and an expiration time. The method can include calculating, by the computing device, from the plurality of dispensing events, an amount of substance dispensed per dispensing event. The method can include select, by the computing device, based on the amount of substance dispensed per dispensing event and the expiration time, a refill amount of the one or more predetermined refill amounts. The method can include providing, by the computing device, via an interactive interface, an indication of the selected the refill amount.

In some embodiments, selecting the refill amount of the one or more predetermined refill amounts includes receiving, by the computing device, from the interactive interface, a first expenditure per unit of the substance and a second expenditure per refill of the pump. Selecting the refill amount of the one or more predetermined refill amounts can further include selecting, by the computing device, the refill amount based on the amount of substance dispensed per dispensing event, the expiration time, the first expenditure, and the second expenditure.

In some embodiments, selecting the refill amount of the plurality of refill amounts includes calculating, by the computing device, the refill amount as proportional to a mean and a standard deviation of the amount of the substance that was dispensed during the plurality of dispensing events. Selecting the refill amount of the plurality of refill amounts can further include identifying, by the computing device, an inverse of a probability density function of the plurality of dispensing events.

In some embodiments, the method can include identifying, by the computing device, based on an expected refill amount of the one or more predetermined refill amounts and the amount of substance dispensed per dispensing event, an expected amount of substance remaining in the pump at a first expected refill time of the substance based on the expected refill amount. The method can include identifying, by the computing device, based on the selected refill amount and the amount of substance dispensed per dispensing event, an estimated amount of substance remaining in the pump at a second expected refill time of the substance based on the selected refill amount. The method can include calculating, by the computing device, a difference between the expected amount of substance remaining and the estimated amount of substance remaining. The method can include providing, by the computing device, via the interactive interface, the difference.

In some embodiments, the method can include generating, by the computing device, an expenditure comparison based on (i) the first expenditure and the difference, and (ii) the second expenditure and a ratio between a first refill frequency of the pump based on the expected amount of substance and a second refill frequency of the pump based on the estimated amount of substance. The method can include providing, by the computing device, via the interactive interface, the expenditure comparison.

In some embodiments, the method can include receiving, by the computing device, via the interactive interface, a preferred refill time of the substance. The method can include identifying, by the computing device, an estimated amount of substance remaining in the pump at the preferred refill time of the substance.

In some embodiments, the method can include identifying, by the computing device, the amount of substance dispensed per dispensing event as a basal rate and a bolus rate of substance dispensing.

In some embodiments, the method can include identifying, by the computing device, each of the one or more predetermined refill amounts of the substance as corresponding to a size of a cartridge with the substance.

In some embodiments, the method can include calculating, by the computing device, a first metric by multiplying the first expenditure by the amount of substance dispensed per dispensing event. The method can include calculating, by the computing device, a second metric by adding the second expenditure to a product of the first expenditure and an estimated amount of substance remaining in the pump at the expiration time of the substance or an expected refill time of the substance, the estimated amount of substance remaining based on a current amount of the substance and the amount of substance dispensed per dispensing event. The method can include identifying, by the computing device, the probability density function of the plurality of dispensing events based on a proportion between a first metric and a second metric.

In some embodiments, the method can include generating, by the computing device, a graph comprising the amount of the substance that was dispensed during each of the plurality of dispensing events. The method can include calculating, by the computing device, a line of best fit of the amount of the substance that was dispensed during each of the plurality of dispensing events. The method can include display, by the computing device, via the interactive interface, the graph having the line of best fit.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1A is a block diagram depicting an embodiment of a network environment comprising local devices in communication with remote devices.

FIG. 1B is a block diagram depicting embodiments of computers useful in connection with the methods and systems described herein.

FIG. 2 is a pictorial representation of an environment for pharmaceutical injection.

FIG. 3 is a block diagram depicting a system configured for pharmaceutical injection supply management configured to manage the pharmaceutical injection environment of FIG. 2, according to one or more embodiments.

FIG. 4A is an exemplary interface for receiving user inputs, according to one or more embodiments.

FIG. 4B is a table of exemplary pump usage data stored by the pharmaceutical injection supply manager, according to one or more embodiments.

FIG. 4C is on a graph of pump usage data stored by the pharmaceutical injection supply manager, according to one or more embodiments.

FIG. 4D is an exemplary interface of selecting pump usage data stored by the pharmaceutical injection supply manager, according to one or more embodiments.

FIG. 4E is a table of an exemplary cost comparison between using average amounts and recommended amounts of pharmaceutical supplies, according to one or more embodiments.

FIG. 4F is an exemplary interface of pump usage data and recommended refill amounts, according to one or more embodiments.

FIG. 5 is a flowchart diagram illustrating a flow for managing pharmaceutical injection supplies, according to one or more embodiments.

FIG. 6 is a flowchart diagram illustrating a method for managing pharmaceutical injection supplies, according to one or more embodiments.

The details of various embodiments of the methods and systems are set forth in the accompanying drawings and the description below.

### DETAILED DESCRIPTION

For purposes of reading the description of the various embodiments below, the following descriptions of the sections of the specification and their respective contents may be helpful:

Section A describes a network environment and computing environment, which may be useful for practicing embodiments described herein.

Section B describes embodiments of systems and methods for pharmaceutical injection supply management.

### A. Computing and Network Environment

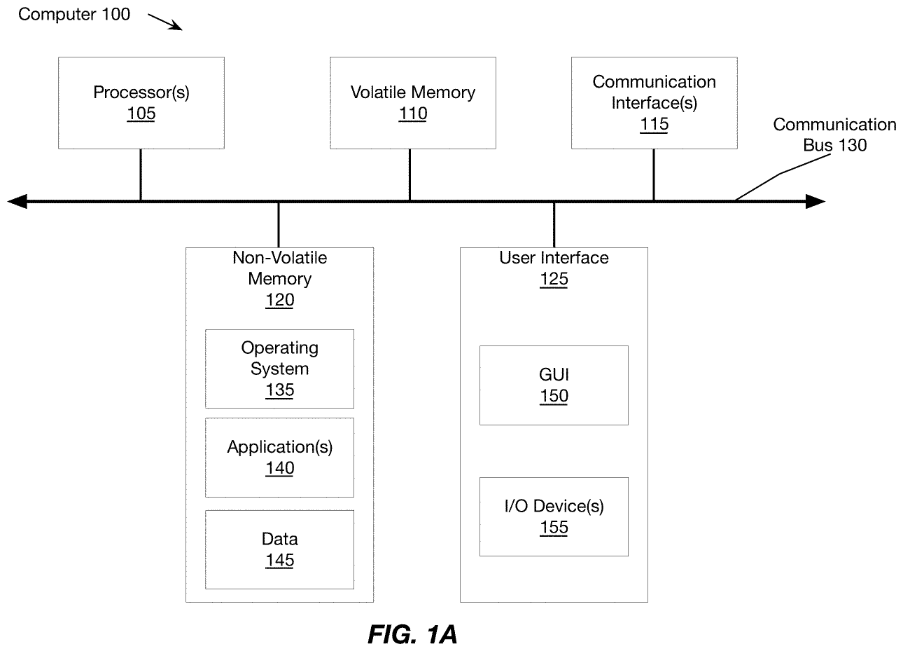

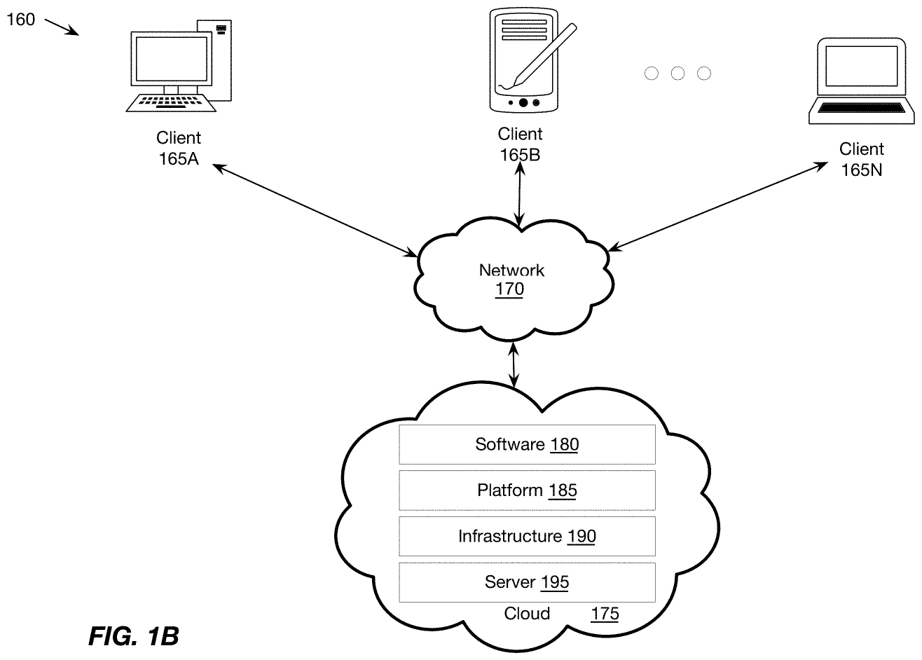

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 1G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud-computing environment is depicted. A cloud-computing environment may provide client 102 with one or more resources provided by a network environment. The cloud-computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms, or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud-based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers, or virtualization resources from large pools, allowing the users to quickly scale up by accessing more resources as needed. Examples of IaaS include AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 may also access SaaS resources through smartphone or tablet applications, including, for example, Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device, or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a management system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by NVidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of a multi-core processor include the AMD PHENOM IIX2, INTEL CORE i5, and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnects bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition, which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable, or provide for the connection and use of multiple display devices 124a-124n by the computing device 100. For example, the computing device 100 may include any type and/or form of video adapter, video card, driver, and/or library to interface, communicate, connect, or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments, software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the multi-dispenser management system 120a. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid-state hybrid drives that combine hard disks with solid-state cache. Some storage device 128 may be non-volatile, mutable, or read-only. Some storage device 128 may be internal and connect to the computing device 100 via a bus 150. Some storage device 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase, and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMAX and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Wash. In other embodiments, the computing device 100 is an eBook reader, e.g. the KINDLE family of devices by Amazon.com, or NOOK family of devices by Barnes & Noble, Inc. of New York City, N.Y.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Systems and Methods for Pharmaceutical Injection Supply Management

Management of pharmaceutical injection supplies can be difficult because users can either load too much of the pharmaceutical injection supply and risk having it expire, or load too little and risk not having enough to satisfy medical needs or having to perform frequent refills. Moreover, performing additional refills can be wasteful because any remaining pharmaceutical injection supply (e.g. part of a vial or supply unit) often needs to be thrown away during the refills.

Implementations of the systems and methods discussed herein provide direction of an optimal amount of pharmaceutical injection supply for the user to load into their dispenser. For example, the pharmaceutical injection supply can be insulin, and the dispenser can be an insulin pump. In particular, the present disclosure can provide, via a mobile application, a recommendation to a pharmaceutical user of the amount of substance to load into their substance pump during a site change. The recommendation can be optimized to minimize substance costs and substance waste, or to accommodate a target replacement date and time.

Substance can be delivered by a commercial pump to a user in a several ways. A steady drip of substance may be referred to as a basal rate, and specific dosage amounts corresponding to a consumption event (e.g. corresponding to consumption of food at a meal) can be referred to as a bolus injection. The source of the substance can be a vial or other container, sometimes referred to as a cartridge, filled with substance by the user and loaded into the pump. The substance can be delivered via a cannula or directly to an injection site. The pump can be held at an injection site for a number of days, depending on the user and the implementation of the pump. To change the injection site, in some implementations, the user can replace materials for the site, cannula, and/or cartridge. Any leftover substance in the cartridge is typically disposed of because it can lose its efficacy over time once loaded into the cartridge or pump, or because the amount of leftover substance is non-zero, but may not be processed by the implementation of the pump.

In some implementations, the application can consider setup costs, substance costs, and substance usage to recommend the optimal amount of substance to load. The cost of a setup can include costs of the materials, such as cartridge, cannula, and site materials, which must be replaced every time there is a new site setup. These costs may be dynamically added or updated by the user and/or a manufacturer or administrator (e.g. over a network). In some implementations, the cost of substance can be the cost of a unit of substance, which can be a standard unit of measure, and may be entered by the user into the app or loaded by an administrator or manufacturer.

In some implementations, the distribution of daily substance usage can include at least three elements: the shape of the usage distribution, the mean, and standard deviation of the total daily amount of substance used by the user. The total daily amount can be defined as the total basal amount delivered plus the total bolus amount delivered. The total basal amount can be the basal rate (units/hour) times the hours the basal rate was delivered over a 24-hour period. The total bolus amount can be the sum of any bolus injections over the same 24-hour period. In some implementations, the distribution shape, mean, and standard deviation can be determined through automated statistical analysis of values extracted from the software that controls the pump.

In some implementations, the application can display a distribution and fitting of data to a statistical distribution of values generated from the mean and standard deviation of daily usage. The application can optimize the value of the site set up to either minimize costs and wasted substance or to give greater control of the duration of the process, based on user preference. For example, if less substance is loaded, then site changes will, on average, be more frequent, which would raise setup costs, but the amount of leftover substance will, on average, be lower, which would lower overage costs. In some implementations, the application can use usage data to identify a balance point between setup costs and overage costs. This application can use a model to balance overage and underage costs to provide a recommended amount of supply in response to uncertain demand. In some implementations, the application can use the model to identify a desired “service level” to translate into an expected duration for a given amount of substance.

In some implementations, the application can use the costs to calculate a critical ratio, which can be defined as

$$C R = \frac{{C}_{u}}{{C}_{u} + {C}_{o}} ,$$

which is between 0 and 1, inclusive. The critical ratio can be the cost of the underage divided by the sum of the cost of overage and cost of underage. The cost of the overage can be the per unit cost of substance multiplied the user's average (mean) daily substance usage. The cost of the overage can be represented as Cₒ. The cost of underage can be the total of all costs associated with a new site prep, the cost of materials, any substance “pre-loaded” into the cannula, and any loaded substance that the pump cannot deliver. The cost of the underage can be represented as Cᵤ. If the users prefer a desired time, in some implementations, the critical ratio can represent a “service level,” which can be the probability that there will be left over substance at the end of the desired duration.

In some implementations, the application can generate the user's multi-day distribution mean and standard deviation from the daily demand information drawn from the user's pump. The duration of the multi-day period can depend on the pump's substance capacity, the user's average substance demand, the maximum time between site changes, and any substance that is loaded but the pump cannot deliver. The period can be one, two, three, four, five, six, or more days, or any other such period. In some implementations, the daily usage can be determined from the pump's activities, as described above. The multi-day values may be the sum of the daily values for the current and previous days covered by this period. The application can perform data transformation as needed, such as based on the best-fit distribution determined by the automated process. In some implementations, the application can calculate the multi-day mean and standard deviation from the final values.

In some implementations, the application can use the multi-day distribution, mean, standard deviation, and user preferences to calculate the amount of substance to be loaded into pump. In some implementations, this may be done by taking the inverse of the cumulative probability density function corresponding to the distributional shape identified at a given probability. The probability can be the probability of using all the substance prior to the end of the multi-day period for a given amount of loaded substance and can be equal to the critical ratio calculated in the first step or the corresponding time preference from the user. In mathematical notation, F (Q) can be the cumulative probability density function for some distributional shape with known mean and standard deviation, where Q is the quantity of substance loaded, F⁻¹(⋅) can be the inverse of that function, and Q* can represent the optimal quantity of substance to be loaded. Using the variables, the application can determine the optimal amount to load as:

$${Q}^{*} = {F}^{- 1} ( C R ) = {F}^{- 1} ( \frac{{C}_{u}}{{C}_{u} + {C}_{o}} )$$

In some implementations, the application can manage the data and calculations relating to substance usage and recommendations. For example, the application can store the data in five categories: “Rec—LN”, “Totals”, “Duration check”, “Basal Rate” and “Bolus.” The “Basal Rate” and “Bolus” categories can include raw data, which can be summarized in the “Totals” categories. The “Rec—LN” category can be known as “Recommendation—Log Normal [Distribution],” which can represent the category of data for generating the recommended amount of substance to load. The “Totals” category can make additional calculations to refine the recommendation. In some implementations, the data can correspond to a single user.

In some implementations, the application can use the data in a model. In some implementations, the application can identify whether the distribution of daily substance usage is lognormal. In some implementations, the application can be preconfigured with anonymous data of substance usage patterns, and/or can perform a statistical ‘best fit’ test on several different distributions with ranges between zero and infinity. In some implementations, the application can identify a best distributional shape for each user and include data that indicates lognormal distributions. The application can distinguish between a tradeoff in any prediction model between the amount of prior data to use and the relevancy of that data to the current use level. For example, the application can use a counterfactual analysis, multiple different lengths of prior data (between 3 and 90 days) for the recommendation. In some implementations, the application can perform a test using, for example, a 7-day rolling average. In some implementations, the application can identify if the amount of substance loaded is different from the amount available for use in the pump and accordingly can identify a measurement error. For example, the application can identify a measurement error of 0.4 mL, or 40 units of substance. The application can incorporate the measurement errors into the recommended load amount. For example, the application can add the measurement error to the recommended amount.

In some implementations, the application can compare the recommended load amount to a user's usage patterns. The comparison can relate to the category in the “Rec—LN” about the expected savings. In some implementations, the application can compare the recommended load to a daily average amount of substance use over a number of desired days. However, the application can consider whether the users exhibit different behaviors such as fully loading the cartridge or loading a minimum allowed amount based on the user's supply of substance, financial condition, and/or health status. In some implementations, the application can identify the cost of substance, cost of site prep materials, cartridge capacity, and time that the pump is on the body. The application can also consider the data assumptions to determine possible savings in substance and money.

In some implementations, the application can include data collection capabilities to automate the process of capturing each substance delivery (basal rate or bolus injection), sum, and transform the data as needed to determine the refill amount. For example, the application can solicit from the user certain information, such as substance cost, site prep cost and goal (optimized cost or target date/time). The application can provide the recommended amount to load during each refill event, as well as comparison data if desired.

In some implementations, the application can calculate the expected savings, in terms of dollars saved and reduction in wasted substance, by calculating the expected amount of money spent on substance waste if the user were to load substance based on their previous usage patterns. The application can provide the recommended load amounts to the user along with the expected cost and pharmaceutical savings that can be calculated from the usage patterns. The application can provide a comparison between loading the optimal amount and loading the average usage amount, a full cartridge, or a user's preference.

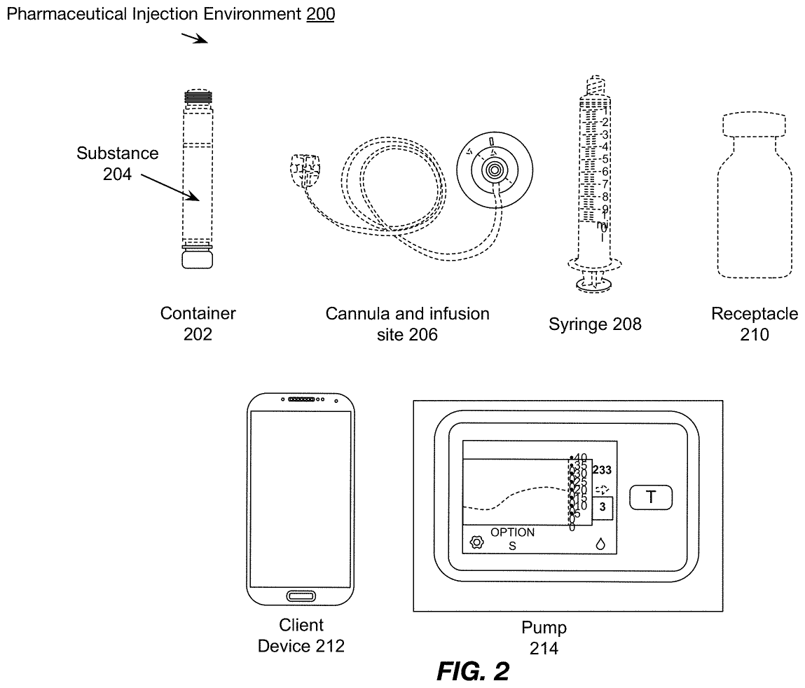

FIG. 2 is a pictorial representation of an environment 200 for pharmaceutical injection. The environment 200 can include a container 202 configured to store substance 204, such as insulin, for refilling into the pump 214. In some embodiments, the container 202 can be a cartridge. The environment 200 can include a cannula and infusion site 206 configured to puncture the user for injecting the substance 204 flowing in the cannula. The environment 200 can include a syringe 208 configured to measuring or injecting the substance. The environment 200 can include a receptacle 210 configured to store the substance 204, such as Lantus 100 U/ml Insulin Solution—10 ml. The environment 200 can include a client device 212 configured to receive or display information relating to pharmaceutical injection supply management. The environment 200 can include a pump 214 configured to dispense the substance. The pump 214 can include a display screen.

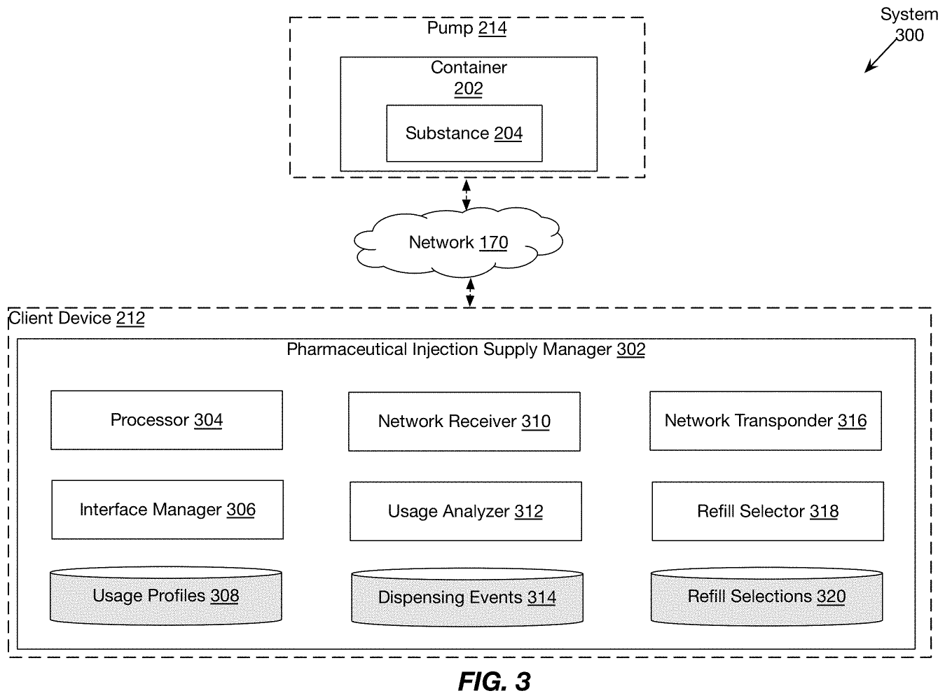

Referring now to FIG. 3, FIG. 3 is a block diagram depicting a system 300 configured for pharmaceutical injection supply management configured to manage the pharmaceutical injection environment of FIG. 2, according to one or more embodiments. In brief overview, the system 300 can include a pump 214 configured to store the container 202 with the substance 204. The system can further include the client device 212 that includes a pharmaceutical injection supply manager 302, which includes a processor 304, an interface manager 306, usage profiles 308, a network receiver 310, a usage analyzer 312, dispensing events 314, a network transponder 316, a refill selector 318, and refill selections 320.

As described in greater detail below, the pharmaceutical injection supply manager 302 can include a processor or plurality of processors 304 configured to execute the components described herein. The interface manager 306 can be configured to provide interfaces (e.g. graphical user interfaces, spoken word interfaces, etc.) to the client device 212. The interface manager 306 can be configured to manage usage profile 308 indicative of expenditures and usage of the substance 204. The network receiver 310 can be configured to communicate with the pump 214 via the network 170. The usage analyzer 312 can be configured to detect the pump 214 dispensing the substance 204. The usage analyzer 312 can be configured to store dispensing events 314 indicating dispensing of the substance 204 by the pump 214. The network transponder 316 can be configured to communicate with the pump 214. The refill selector 318 can be configured to select an amount of substance 204 to refill into the pump 214. The refill selector 318 can be configured to store the selected amounts as the refill selections 320.

The pharmaceutical injection supply manager 302 (include the processor 304, the interface manager 306, the usage profiles 308, the network receiver 310, the usage analyzer 312, the dispensing events 314, the network transponder 316, the refill selector 318, and the refill selections 320) can be implemented using components described in connection with FIGS. 1A and 1B. Each of these elements or entities can include any application, program, library, script, task, service, process or any type and form of executable instructions executing on hardware of the various components in the pharmaceutical injection supply manager 302 (include the processor 304, the interface manager 306, the usage profiles 308, the network receiver 310, the usage analyzer 312, the dispensing events 314, the network transponder 316, the refill selector 318, and the refill selections 320). The hardware includes circuitry such as one or more processors 304 in one or more embodiments.

The interface manager 306 can be configured to provide interactive controls and information to the client device 212, including graphical user interfaces, command line interfaces, voice-based command interfaces, etc. The interface manager 306 can be configured to request or receive user information, such as substance 204 cost, refill cost (e.g., site prep costs) and goals (optimized costs or refill at a preferred date or time). The interface manager 306 can be configured to include data collection capabilities to automate the process of detecting each substance 204 dispensing (basal rate or bolus injection).

The interface manager 306 can be configured to manage usage profile 308 indicative of expenditures and usage of the substance 204 for each user. Substance 204 can be delivered by a commercial pump 214 to a user in a several ways. For example, a steady drip of substance 204 may be referred to as the basal rate, and specific dosage amounts (e.g. corresponding to food consumption) can be referred to as a bolus injection. The source of substance 204 can be a vial, container, or cartridge, filled with substance 204 by the user and loaded into the pump 214. The substance 204 can be delivered via the cannula 206 to an injection site. The pump 214 can be held at an injection site for a number of days that depends on the user and the implementation of the pump 214. To change the injection site, in some implementations, the user can replace materials for the site, cannula, and cartridge. Any leftover substance 204 in the container 202 may be disposed of (e.g. due to loss of efficacy, difficulty in recovering small amounts of the substance from the container, etc.).

In some implementations, the interface manager 306 can be configured to receive expenditures. In some implementations, the interface manager 306 can be configured to receive expenditures via an interface from the user. In some embodiments, the interface manager 306 can be configured to receive, from the interactive interface, a first expenditure per unit of the substance 204 and a second expenditure per refill of the pump 214. The first expenditure can correspond to a cost per unit of the substance 204. For example, the first expenditure can identify a cost per unit of substance 204. The second expenditure can identify a cost to refill the pump 214 with the substance 204. For example, the second expenditure can identify a cost to site prep (e.g., change caps, needles, pump 214 locations, etc.) for the refill of the pump 214.

The interface manager 306 can be configured to receive or identify setup costs, substance 204 costs, and substance 204 usage to recommend the amount of substance 204 to refill the pump 214. The cost of a setup can include costs of the materials, such as cartridge, cannula, and site materials, which must be replaced every time there is a new site setup. The interface manager 306 can be configured to receive these costs from the user, and update them if there are any changes. The cost of substance 204 can be the cost of a unit of substance 204, which can be a standard unit of measure, and is likewise entered by the user into the app.

The interface manager 306 can be configured to provide the refill amount to load during each refill event. After the refill selector 318 selects the refill amount, the interface manager 306 can be configured to provide an indication of the selected refill amount. The interface manager 306 can be configured to provide the indication via a graphical user interface. In some embodiments, the interface manager 306 can be configured to display the selected refill amount on a display of the pump 214.

In some embodiments, the interface manager 306 can be configured to display comparisons of expenditures between the refill amount and a generic amount.

In some embodiments, the network receiver 310 can be configured to communicate with the pump 214 via the network 170 to detect dispensing events. The network receiver 310 can be configured to receive dispensing events indicative of the pump 214 dispensing the substance 204.

In some embodiments, the usage analyzer 312 can be configured to detect, via the network receiver 310, configured to receive dispensing activity of a pump 214 communicatively coupled to the network receiver, dispensing events. Based on the dispensing events, the network receiver 310 can be configured to identify an amount of a substance 204 that was dispensed or an expiration time of the substance 204. The usage analyzer 312 can be configured to identify or extract the timestamp, the amount, or the expiration time from each of the dispensing events. The usage analyzer 312 can be configured to identify predetermined refill amounts for the pump 214. For example, the usage analyzer 312 can be configured to identify that the pump 214 can be refilled with 50 units of 100 units of substance 204.

In some embodiments, the usage analyzer 312 can be configured to store or identify dispensing events 314 indicating dispensing of the substance 204 by the pump 214. The usage analyzer 312 can be configured to store a distribution of daily substance 204 usage, which can include at least three elements: the shape of the usage distribution, the mean, and standard deviation of the total daily amount of substance 204 used by the user. The total daily amount can be defined as the total basal amount delivered plus the total bolus amount delivered. The total basal amount can be the basal rate (e.g. units/hour, though other time periods are possible) times the hours the basal rate was delivered over a 24-hour period (similarly, other time periods may be utilized). The total bolus amount can be the sum of any bolus injections over the same 24-hour period or other duration. The distribution shape, mean, and standard deviation can be determined through automated statistical analysis of values extracted from the software that controls the pump 214. The usage analyzer 312 can be configured to display a distribution and fitting of data to a statistical distribution of values generated from the mean and standard deviation of daily usage.

The usage analyzer 312 can be configured to calculate an amount of substance 204 dispensed. The usage analyzer 312 can be configured to identify or extract, from the plurality of dispensing events, an amount of substance 204 dispensed. The usage analyzer 312 can be configured to identify a timestamp associated with each of the dispensing events. Based on the amount of substance 204 dispensed and the timestamp, the usage analyzer 312 can be configured to calculate, identify, or extract an amount of substance 204 dispensed per dispensing event. The usage analyzer 312 can be configured to identify the amount of substance 204 dispensed per dispensing event as a basal rate and a bolus rate of substance 204 dispensing. In some embodiments, the usage analyzer 312 can be configured to receive the amount of substance 204 dispensed from the pump 214.

The usage analyzer 312 can be configured to generate the user's multi-day distribution mean and standard deviation from the daily usage information received from the user's pump 214. The duration of the multi-day period can depend on the pump 214's substance 204 capacity, the user's average substance 204 demand, the maximum time between site changes, and any substance 204 that is loaded but the pump 214 cannot deliver. The period can be one, two, three, four, five, six, or more days. The daily usage can be determined from the pump 214's dispensing activities, as described above. The multi-day values can be the sum of the daily values for the current and previous days covered by this period. The usage analyzer 312 can be configured to perform data transformation as needed, such as based on the best-fit distribution. The usage analyzer 312 can be configured to calculate the multi-day mean and standard deviation of substance 204 dispensing.

In some embodiments, the usage analyzer 312 can be configured to identify whether the substance 204 is expired. The usage analyzer 312 can be configured to receive an expiration time of the substance 204 from the pump 214 or via the graphical user interface. For example, the usage analyzer 312 can be configured to receive a date and/or time at which the substance 204 in the pump 214 cannot be dispensed. The usage analyzer 312 can be configured to store the expiration time. To identify or determine whether the substance 204 is expired, in some embodiments, the usage analyzer 312 can be configured to compare the expiration time to the current time. In some embodiments, the usage analyzer 312 can be configured to receive, from the pump 214, a status indicating whether the substance 204 is expired. The usage analyzer 312 can be configured to store the status.

If the substance 204 is not expired, in some implementations, the usage analyzer 312 can be configured to identify whether an amount of substance 204 remaining satisfies a threshold. The usage analyzer 312 can be configured to identify the amount of substance 204 remaining in the pump 214. By identifying the amount of substance 204 that was dispensed the usage analyzer 312 can be configured to identify or calculate the amount of substance 204 remaining in the pump 214. For example, if the pump 214 was loaded with 100 units and the pharmaceutical injection supply manager detects five dispensing events of 10 units each, the usage analyzer 312 can be configured to calculate that the pump 214 has 50 units of substance 204 remaining.

In some implementations, the usage analyzer 312 can be configured to identify or receive the threshold corresponding to an amount of substance 204 remaining in the pump 214 at which the pump 214 is to be refilled. For example, usage analyzer 312 can be configured to identify that the threshold is 50 units. In some implementations, the usage analyzer 312 can be configured to select or suggest to refill the pump 214 when the amount of substance 204 remaining satisfies (e.g., is less than) the threshold. Based on the previous example, the usage analyzer 312 can be configured to select or suggest refilling the pump 214 when the amount of substance 204 remaining is less than 50 units. In some implementations, the usage analyzer 312 can be configured to receive the threshold via the interface. The usage analyzer 312 can be configured to modify the threshold based on dispensing events. For example, the usage analyzer 312 can be configured to increase the threshold responsive to identifying that the pump 214 frequently ran out of substance 204 at the previous threshold. Conversely, the usage analyzer 312 can be configured to decrease the threshold responsive to identifying too many refills of the pump 214.

If the amount satisfies the threshold, the usage analyzer 312 can be configured to calculate a critical ratio. For example, if the amount of substance 204 remaining is 45 units but the threshold is 50 units, the usage analyzer 312 can be configured to calculate or identify the critical ratio. The usage analyzer 312 can be configured to calculate or identify the critical ratio to identify a proportion between the expenditures and the amount of substance 204 dispensed. The usage analyzer 312 can be configured to use the critical ratio to identify an optimal amount of substance 204 to refill into the pump. The usage analyzer 312 can be configured to calculate or identify the critical ratio as

$$CR = \frac{{C}_{u}}{{C}_{u} + {C}_{o}} ,$$

which is between 0 and 1, inclusive. The critical ratio can be the cost of the underage divided by the sum of the cost of overage and cost of underage.

In some implementations, to calculate the critical ratio, the usage analyzer 312 can be configured to calculate or identify a mean and a standard deviation of the amount of the substance 204 that was dispensed during the dispensing events. The usage analyzer 312 can be configured to calculate or identify the mean and the standard deviation from the each of the amounts of substance 204 dispensed during the dispensing events. The usage analyzer 312 can be configured to use the mean to identify an average amount of substance 204 dispensed per dispensing event.

Based on the mean and standard deviation, the usage analyzer 312 can be configured to generate the user's multi-day distribution from the dispensing events drawn from the pump 214. The usage analyzer 312 can be configured to identify a duration of the multi-day period based on the pump's substance 204 capacity, the user's average dispensing usage, the maximum time between site changes, and any substance 204 that is loaded but the pump 214 cannot deliver. The period can be one, two, three, four, five, six, or more days, or any other such time period. The hourly or daily usage can be determined from the pump's activities, as described above. For example, the multi-day values are the sum of the daily values for the current and previous days covered by this period. The usage analyzer 312 can be configured to perform data transformation as needed, such as based on the best-fit distribution determined by the automated process.

Based on the calculated or identified mean and standard deviation, in some implementations, the usage analyzer 312 can be configured to calculate or identify a first metric based on a first expenditure. The first metric corresponds to the underage expenditure, which indicates the cost of discarding the substance 204 that remains in the pump 214 during the refill combined with the cost of refilling the pump 214. For example, the cost of underage can be the total of all costs associated with a new site prep, the cost of materials, any substance 204 loaded in the cannula 206, and any loaded substance 204 that the pump 214 cannot dispense. The cost of the underage can be represented as Cᵤ. The usage analyzer 312 can be configured to calculate or identify a first metric by multiplying the first expenditure (e.g., per unit cost) by the amount of substance 204 dispensed per dispensing event.

Based on the calculated or identified mean and standard deviation, the usage analyzer 312 can be configured to calculate or identify a second metric based on a second expenditure. The second metric corresponds to the overage expenditure, which indicates the per unit cost of the substance 204. The cost of the overage can be the per unit cost of substance 204 multiplied by the user's average (mean) daily substance 204 usage. The cost of the overage can be represented as Cₒ. The usage analyzer 312 can be configured to calculate or identify a second metric by adding the second expenditure (e.g., site prep cost) to a product of the first expenditure and an estimated amount of substance 204 remaining in the pump at the expiration time of the substance 204. For example, if the refill amount is 500 units, the amount dispensed per dispensing event is 50, the pharmaceutical injection supply manager expects two dispensing events each day, and substance 204 will expire in 4 days, the usage analyzer 312 can be configured to calculate that the amount of substance 204 remaining at the expiration time will be 100 units.

In some embodiments, the usage analyzer 312 can be configured to calculate or identify the second metric by adding the second expenditure (e.g., site prep cost) to a product of the first expenditure and an estimated amount of substance 204 remaining in the pump 214 at an expected refill time of the substance 204. In some embodiments, the expected refill time is when the pump 214 cannot dispense the necessary amount of substance 204. The usage analyzer 312 can be configured to identify or calculate the estimated amount of substance 204 remaining based on a current amount of the substance 204 and the amount of substance 204 dispensed per dispensing event. For example, if the refill amount is 225 units and the amount dispensed per dispensing event is 50, the usage analyzer 312 can be configured to calculate that the amount of substance 204 remaining at the refill time will be 25 units.

In some embodiments, the expected refill time is when the user of the pump 214 prefers to refill the pump 214. The usage analyzer 312 can be configured to receive the expected refill time via the interface. For example, the expected refill time can be in the morning when the user prefers to refill the pump 214. For example, if the amount of substance 204 in the pump 214 is 500 units, the amount dispensed per dispensing event is 150, and the pharmaceutical injection supply manager expects two dispensing events each day, the usage analyzer 312 can be configured to calculate that the expected amount of substance 204 remaining at the preferred refill time will be 200 units.

Since the amount of substance 204 dispensed varies over time, in some embodiments, the usage analyzer 312 can be configured to calculate a probability density function for the amount of substance 204 dispensed. In some embodiments, based on the mean, standard deviation, and the amounts dispensed, the usage analyzer 312 can be configured to calculate or generate a probability density function for the dispensing events. Based on the probability density function, the usage analyzer 312 can be configured to manage or store probabilities for amounts of substance 204 dispensed by the pump 214. The usage analyzer 312 can be configured to receive the amount dispensed as the input and output the probability of dispensing that amount. For example, if four dispensing events indicate that the pump 212 dispensed 10 units, 15 units, 20 units, and 25 units, then the usage analyzer 312 can be configured to use the probability density function to identify that the probability of dispensing 10 units is 0.25.

The usage analyzer 312 can be configured to identify or calculate an inverse of the probability density function to identify the amount dispensed at a given probability. In some embodiments, the usage analyzer 312 can be configured to calculate the inverse based on the mean and the standard deviation. Based on the inverse, the usage analyzer 312 can be configured to transform the probabilities in the probability density function to the amount dispensed. To calculate the amount dispensed associated with the given probability, the usage analyzer 312 can be configured to receive the probability as the input and output the amount dispensed. For example, if the pump 214 dispenses 10 units, 15 units, 20 units, and 25 units, the usage analyzer 312 can be configured to receive a probability of 0.25 as an input and use the inverse to identify that the amount dispensed is 10 units, which indicates that there is a 25% chance of dispensing up to 10 units. Similarly, the usage analyzer 312 can be configured to receive a probability of 0.75 as an input and use the inverse to identify that the amount dispensed is 20 units, which includes the dispensing events of 10, 15, and 20 units and indicates that there is a 75% chance of dispensing up to 20 units.

In some embodiments, the usage analyzer 312 can be configured to use the multi-day distribution, mean, standard deviation, and user preferences to calculate the amount of substance 204 to be refilled. The usage analyzer 312 can be configured to calculate the inverse of the cumulative probability density function corresponding to the distributional shape identified at a given probability. The probability can be the probability of using all the substance 204 prior to the end of the multi-day period for a given amount of loaded substance 204 in the pump 214. The usage analyzer 312 can be configured to calculate the probability to be equal to the critical ratio or the corresponding time preference from the user. In mathematical notation, the usage analyzer 312 can be configured to calculate F(Q) as the cumulative probability density function for a distributional shape with known mean and standard deviation, where Q is the quantity of substance 204 loaded, F⁻¹ (⋅) can be the inverse of that function, and Q* can represent the optimal quantity of substance to be loaded. Using the variables, the usage analyzer 312 can be configured to calculate can calculate the optimal amount to load as:

$${Q}^{*} = {F}^{- 1} ( C R ) = {F}^{- 1} ( \frac{{C}_{u}}{{C}_{u} + {C}_{o}} )$$

The usage analyzer 312 can be configured to provide the data into a model. The usage analyzer 312 can be configured to identify that the distribution of daily substance 204 usage is lognormal. The usage analyzer 312 can be configured to be preconfigured with anonymous data of substance 204 usage patterns. The usage analyzer 312 can be configured to perform a statistical ‘best fit’ test on several different distributions with ranges between zero and infinity. The usage analyzer 312 can be configured to identify a distributional shape for each user and include data that indicates the distributional shape, such as a lognormal distribution. The usage analyzer 312 can be configured to distinguish between a tradeoff in any prediction model between the amount of prior data to use and the relevancy of that data to the current use level. For example, the usage analyzer 312 can be configured to use a counterfactual analysis, multiple different lengths of prior data (between 3 and 90 days) for the recommended refill amount. The usage analyzer 312 can be configured to perform a test using, for example, a 7-day rolling average. The usage analyzer 312 can be configured to identify if the amount of substance 204 loaded is different from the amount available for use in the pump. Therefore, the usage analyzer 312 can be configured to identify a measurement error. For example, the usage analyzer 312 can be configured to identify a measurement error of 0.4 mL, or 40 units of substance 204. The usage analyzer 312 can be configured to incorporate the measurement errors into the recommended load amount. For example, the usage analyzer 312 can be configured to add the measurement error to the recommended amount.

Based on the inverse of the probability density function and the critical ratio, the usage analyzer 312 can be configured to calculate or identify the refill amount. The refill amount can be an optimal amount of substance 204 to load into the pump to balance between wasting substance 204 and wasting resources for refilling. For example, the usage analyzer 312 can be configured to identify or calculate a distribution and fitting of data to a statistical distribution of values generated from the mean and standard deviation of dispensing usage.

In some embodiments, the usage analyzer 312 can be configured to calculate a value of the site set up to either minimize costs and wasted substance 204 or to give greater control of the duration of the process, based on user preference. For example, if less substance 204 is loaded, then site changes will, on average, be more frequent, which would raise setup costs, but the amount of leftover substance 204 will, on average, be lower, which would lower overage costs. The usage analyzer 312 can be configured to use usage data to identify a balance point between setup costs and overage costs. This usage analyzer 312 can be configured to use a model to balance overage and underage costs to provide a recommended refill amount.

The network transponder 316 can be configured to communicate with the pump 214. For example, the usage analyzer 312 can be configured to use the network transponder 316 to transmit usage information to the pump 214. In another example, the refill selector 318 can be configured to use the network transponder 316 to transmit the selected refill amount to the pump 214

In some embodiments, the refill selector 318 can be configured to select an amount of substance 204 to refill into the pump 214. The refill selector 318 can be configured to optimize the value of the site set up to either minimize costs and wasted substance 204 or to give greater control of the duration of the process, based on user preference. For example, if less substance 204 is loaded, then site changes will, on average, be more frequent, which would raise setup costs, but the amount of leftover substance 204 will, on average, be lower, which would lower overage costs. The refill selector 318 can be configured to use usage data to identify a balance point between setup costs and overage costs. The refill selector 318 can be configured to use a model to balance overage and underage costs to provide a recommended amount of supply in response to uncertain demand. The refill selector 318 can be configured to apply the model to identify an expected duration without refills for a given amount of substance 204.

In some embodiments, the refill selector 318 can be configured to identify whether an overage expenditure exceeds an underage expenditure. The refill selector 318 can be configured to identify that the overage expenditure exceeds the underage expenditure if the critical ratio is between 0 and 0.5. For example, the formula for the critical ratio will approach 0 as the Cₒ increases relative to the Cₒ. If the overage expenditure exceeds the underage expenditure, the refill selector 318 can be configured to select a small refill amount. By identifying that the overage expenditure exceeds the underage expenditure, the refill selector 318 can be configured to identify that the cost of refilling the pump 214 is less the cost of the substance 204. When the cost of refilling is less than the cost of the substance 204, it would be beneficial to refill the pump 214 often with small quantities to minimize wasting the more expensive substance 204 during refills or due to expiration of the substance 204. Therefore, the refill selector 318 can be configured to select a small refill amount. Since the amount of substance 204 dispensed varies over time, the refill selector 318 can be configured to calculate the inverse of the probability density function to calculate the exact refill amount.

In some embodiments, the refill selector 318 can be configured to identify predetermined refill amounts of the substance 204. The refill selector 318 can be configured to receive the predetermined refill amounts from the pump 214, or the refill selector 318 can be configured to identify the predetermined refill amounts based on the dispensing events. For example, the refill selector 318 can be configured to identify frequent increases in the amount of substance 204 remaining. For example, the refill selector 318 can be configured to identify frequent increases in 100, 200, or 300 units. The increases can correspond to the predetermined refill amounts. The predetermined refill amounts can correspond to a size of a container 202 with the substance 204. For example, the containers 202 can be 100, 200, or 300 units.

In some embodiments, the refill selector 318 can be configured to select the size of the container 202 based on the selected refill amount. For example, if the selected refill amount is 105 units and the container 202 sizes are 100 units and 200 units, the refill selector 318 can be configured to select the 100-unit container 202.

If the overage expenditure does not exceed the underage expenditure, the refill selector 318 can be configured to identify whether the underage expenditure exceeds the overage expenditure. The refill selector 318 can be configured to identify that the underage expenditure exceeds the overage expenditure if the critical ratio is between 0.5 and 1. For example, the formula for the critical ratio will approach 1 as the Cᵤ increase relative to the Cₒ. If the underage expenditure exceeds the overage expenditure, the refill selector 318 can be configured to select a small refill amount. By identifying that the underage expenditure exceeds the overage expenditure, the refill selector 318 can be configured to identify that the cost of refilling the pump 214 is more than the cost of the substance 204. When the cost of refilling is more than the cost of the substance 204, it would be beneficial to refill the pump 214 often with large quantities. Even though refilling with large quantities may cause more waste of the substance 204 during refills or because of expiration, this approach can be less expensive because the cost of the refills exceeds the cost of the discarded substance 204. Therefore, the refill selector 318 can be configured to select a large refill amount. Since the amount of substance 204 dispensed varies over time, the refill selector 318 can be configured to calculate the inverse of the probability density function to calculate the exact refill amount.

If the underage expenditure does not exceed the overage expenditure and the overage expenditure does not exceed the underage expenditure, the refill selector 318 can be configured to select any refill amount. For example, the formula for the critical ratio will approach 0.5 if the Cᵤ equals the Cₒ. By identifying that the overage expenditure equals the underage expenditure, the refill selector 318 can be configured to identify that the cost of refilling the pump 214 is equal to the cost of the substance 204. When the cost of refilling is equal to the cost of the substance 204, the user can refill the pump 214 with any amount of substance 204. For example, the refill selector 318 can be configured to select a refill amount that correspond to the average (mean) amount of substance refilled.

In some embodiments, the refill selector 318 can be configured to store the selected amounts as the refill selections 320. For example, the refill selector 318 can be configured to store refill selections 320 after each refill of the pump 214 with the substance 204.

Based on the stored refill selections 320, the refill selector 318 can be configured to compare between refilling the selected refill amount and an expected refill amount. For example, the refill selector 318 can be configured to compare the recommended load amount to a user's usage patterns. The refill selector 318 can be configured to compare the recommended load to a daily average amount of substance 204 use over a number of desired days. However, the refill selector 318 can be configured to consider whether the users exhibit different behaviors such as fully loading the container 202 or loading a minimum allowed amount based on the user's supply of substance 204, financial condition, and/or health status. The refill selector 318 can be configured to identify the cost of substance 204, cost of site prep materials, container 202 capacity, and time that the pump 214 is on the body. The refill selector 318 can be configured to consider the data assumptions to determine possible savings in substance 204 and money.

In some embodiments, the refill selector 318 can be configured to identify a maximum refill amount, a mean refill amount (e.g., the amount the user typically refills), or a minimum refill amount for the value of the expected refill amount. The maximum refill amount can be the greatest amount of substance 204 to refill into the pump 214. The expected refill amount can be the largest container 202 size of the substance 204. The minimum refill amount can be the least amount of substance 204 to refill into the pump 214. The refill selector 318 can be configured to use the comparison to identify the benefits of refilling the selected amount instead of the expected amount (which can be associated with waste of the substance 204). To make the comparison between refilling the selected refill amount and the expected refill amount, the refill selector 318 can be configured to calculate a difference between the expected amount of substance 204 remaining and the estimated amount of substance 204 remaining.

In some embodiments, to calculate the estimated amount of substance 204 remaining responsive to selecting the selected refill amount, the refill selector 318 can be configured to calculate, identify, or retrieve the amount of substance 204 dispensed per dispensing event. For example, if the selected refill amount is 300 units and the amount dispensed per dispensing event is 150, the refill selector 318 can be configured to calculate that the estimated amount of substance 204 remaining is 0 units. In some embodiments, the refill selector 318 can be configured to calculate or identify the estimated amount of substance 204 remaining based on an expected refill time. In some embodiments, the refill selector 318 can be configured to receive the expected refill time via the graphical user interface or another interface. For example, the expected refill time can be in the morning when the user prefers to refill the pump 214. For example, if the selected refill amount is 300 units, the amount dispensed per dispensing event is 150, and the pharmaceutical injection supply manager expects two dispensing events each day, the refill selector 318 can be configured to calculate that the estimated amount of substance 204 remaining at the preferred refill time will be 0 units.

In some embodiments, the refill selector 318 can be configured to calculate or identify the expected amount of substance 204 remaining based on the amount of substance 204 dispensed per dispensing event. For example, if the expected refill amount is 500 units and the amount dispensed per dispensing event is 150, the refill selector 318 can be configured to calculate that the expected amount of substance 204 remaining is 50 units (at which point the pump 214 will not have enough substance 204 for a dispensing event). In some embodiments, the refill selector 318 can be configured to calculate or identify the expected amount of substance 204 remaining based on an expected refill time. The refill selector 318 can be configured to receive the expected refill time via the graphical user interface. For example, the expected refill time can be in the morning when the user prefers to refill the pump 214. For example, if the expected refill amount is 500 units, the amount dispensed per dispensing event is 150, and the pharmaceutical injection supply manager expects two dispensing events each day, the refill selector 318 can be configured to calculate that the expected amount of substance 204 remaining at the preferred refill time will be 200 units.

In some embodiments, the refill selector 318 can be configured to calculate or identify the difference between the expected amount of substance 204 remaining and the estimated amount of substance 204 remaining. In the aforementioned example, the refill selector 318 can be configured to calculate that the expected amount of substance 204 remaining would be 200 units more than the estimated amount of substance 204 remaining. The refill selector 318 can be configured to identify that the selected refill amount avoids discarding 200 units of the substance 204.

Based on the expected refill amount, the selected refill amount, and their respective amounts of substance 204 remaining, the refill selector 318 can be configured to compare a frequency of refills. For example, if the expected refill amount is 500 units and the amount dispensed per dispensing event is 150, then the refill selector 318 can be configured to calculate that the frequency of refills would be one per three dispensing events. Likewise, if the selected refill amount is 300 units and the amount dispensed per dispensing event is 150, then the refill selector 318 can be configured to calculate that the frequency of refills would be one per two dispensing events. Based on the aforementioned examples, the refill selector 318 can be configured to calculate that the selected refill amount is associated with a 50% increase in the frequency of refills (despite the expected refill amount having an extra 67% of substance 204).

Based on the frequency of refills and the amount of substance 204 expected to remain for discarding at each refill, the refill selector 318 can be configured to calculate or identify a cost of the expected refill amount and the selected refill amount. For example, the refill selector 318 can be configured to divide a summation of the cost of the refills and the cost of the discarded substance 204 by the amount of substance 204 refilled to calculate a cost per unit of substance 204. The cost of the discarded substance 204 can be the first expenditure and the cost of the refills can be the second expenditure. The refill selector 318 can be configured to calculate the cost per unit of substance 204 for the expected refill amount and the selected refill amount. In some embodiments, the expected refill amount and the selected refill amount can generate or identify an expenditure comparison based on (i) the first expenditure and the difference, and (ii) the second expenditure and a ratio between a first refill frequency of the pump based on the expected amount of substance 204 and a second refill frequency of the pump based on the estimated amount of substance 204.

In some embodiments, the refill selector 318 can be configured to provide the comparison via the interface manager 306. The comparison can include the difference between the selected refill amount and the expected refill amount, the minimum refill amount, or an average refill amount. In some embodiments, the refill selector 318 can be configured to provide the comparison via the interface. In some embodiments, the refill selector 318 can be configured to provide the difference to the display of the pump 214.

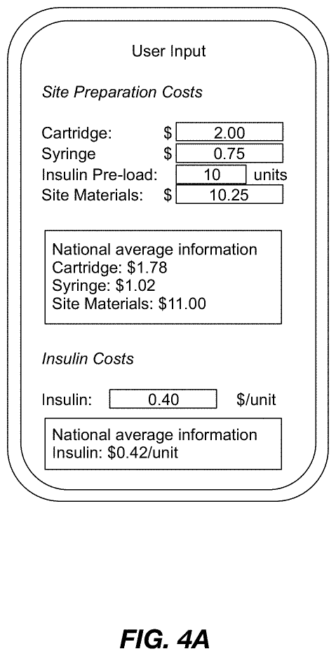

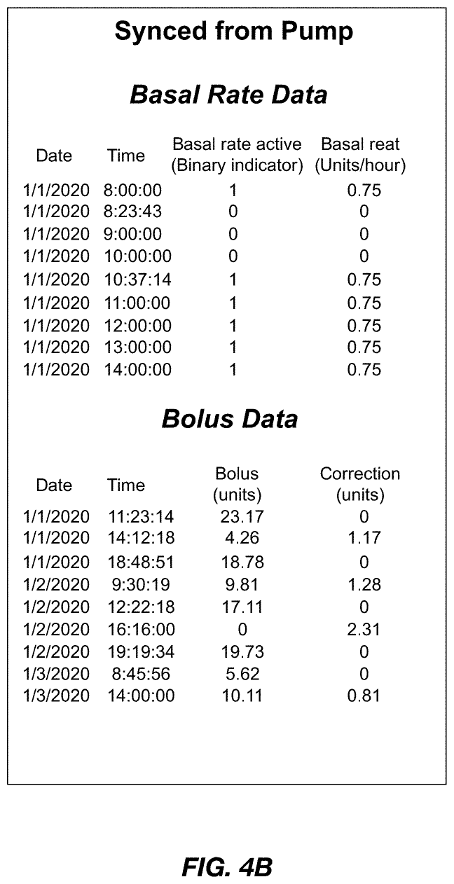

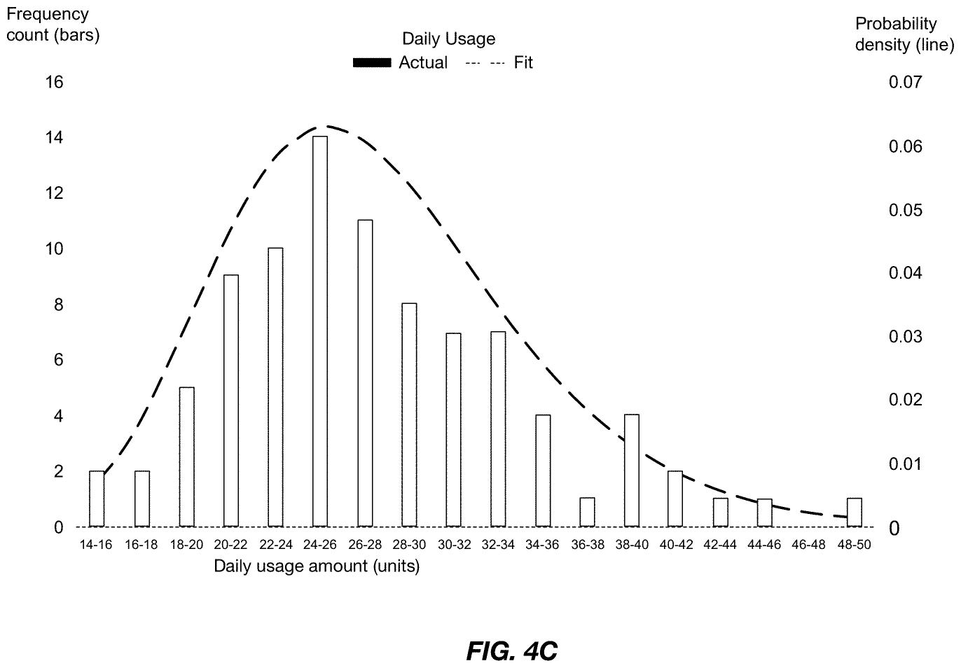

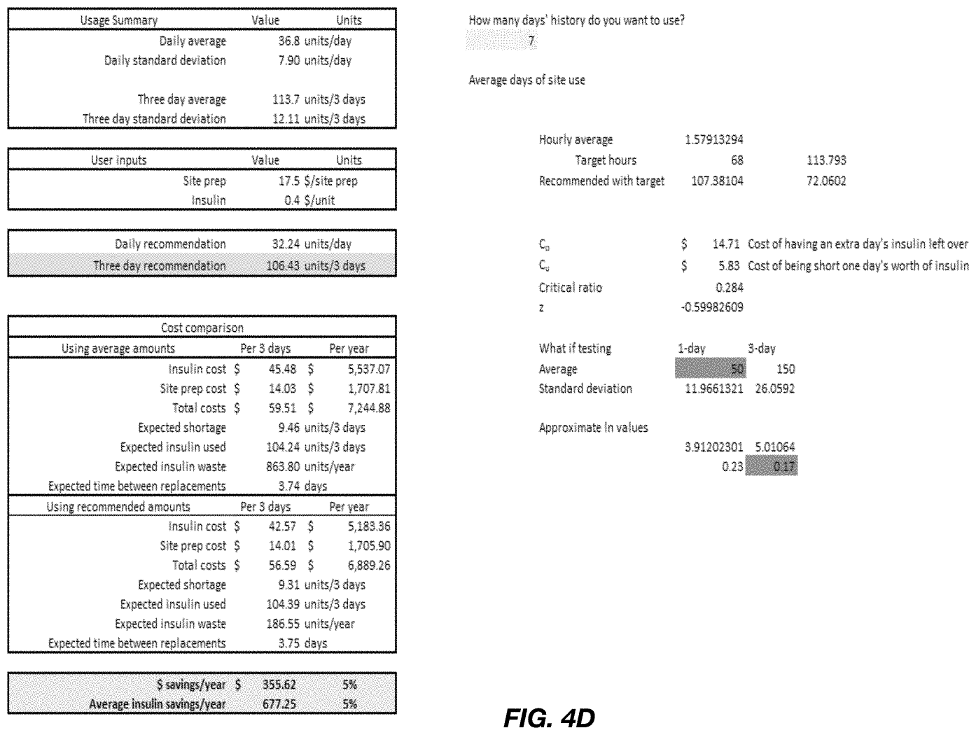

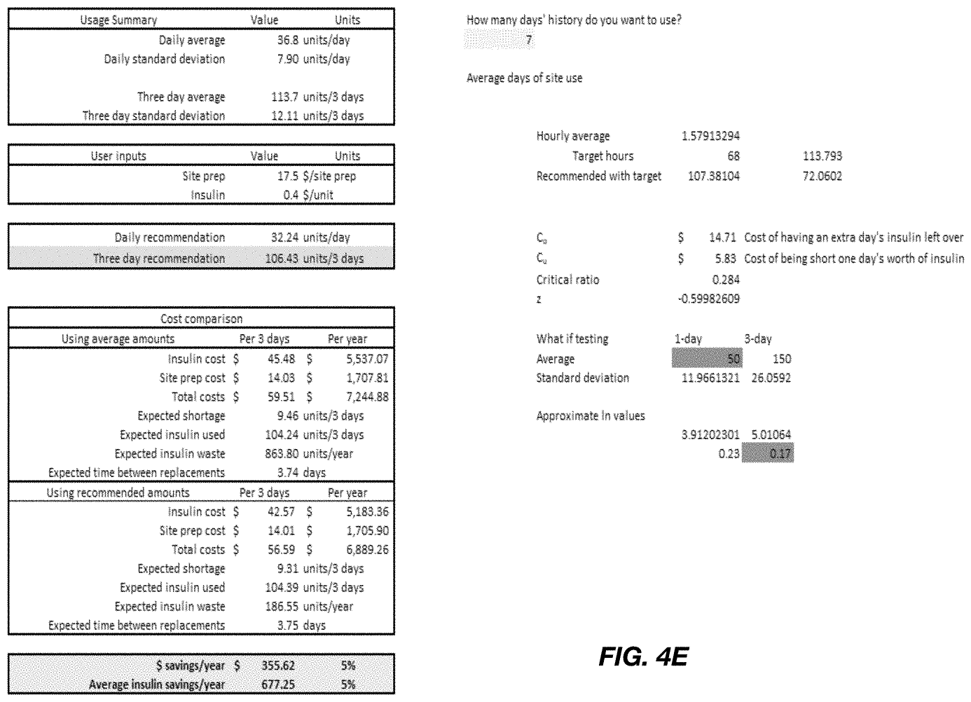

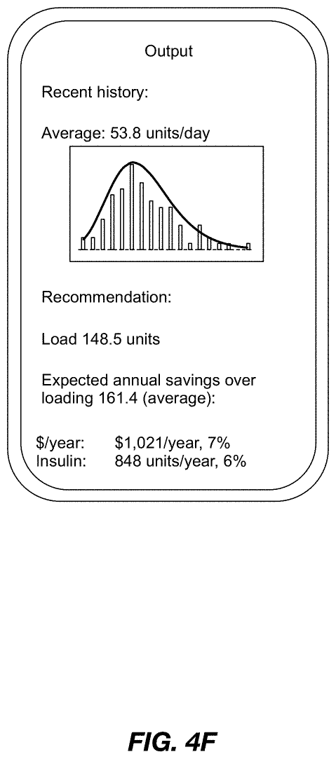

FIG. 4A is an exemplary interface for receiving user inputs, according to one or more embodiments. FIG. 4B is a table of exemplary pump usage data stored by the pharmaceutical injection supply manager, according to one or more embodiments. FIG. 4C is on a graph of pump usage data stored by the pharmaceutical injection supply manager, according to one or more embodiments. FIG. 4D is an exemplary interface of selecting pump usage data stored by the pharmaceutical injection supply manager, according to one or more embodiments. FIG. 4E is a table of an exemplary cost comparison between using average amounts and recommended amounts of pharmaceutical supplies, according to one or more embodiments. FIG. 4F is an exemplary interface of pump usage data and recommended refill amounts, according to one or more embodiments.

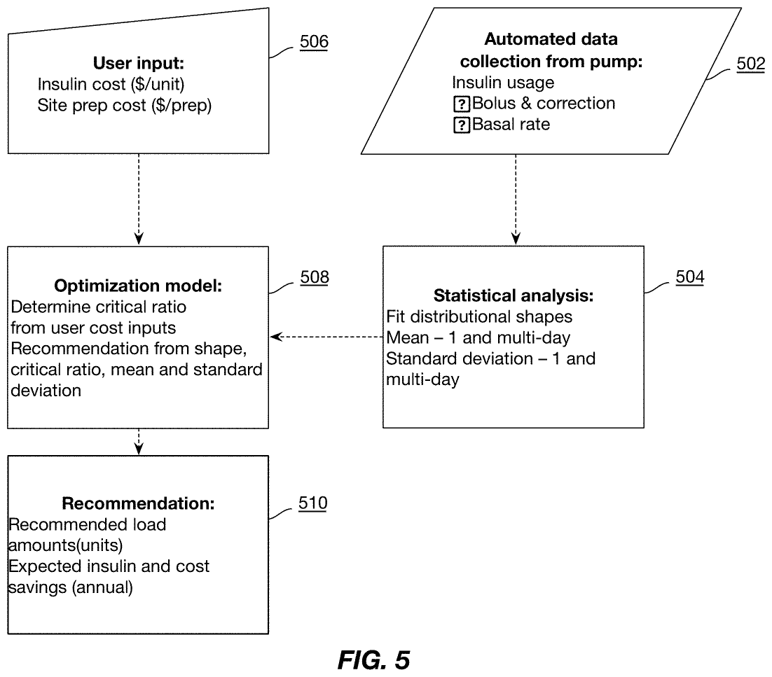

FIG. 5 is a flowchart diagram illustrating a flow 500 for managing pharmaceutical injection supplies. The pharmaceutical injection supply manager 302 can perform the flow 500. The pharmaceutical injection supply manager can automatically collect data from the pump (e.g., pump 214) (STEP 502). The pharmaceutical injection supply manager can detect dispensing events (STEP 504). The pharmaceutical injection supply manager can calculate an amount of substance dispensed (STEP 506). The pharmaceutical injection supply manager can select a refill amount (STEP 508). The pharmaceutical injection supply manager can provide an indication of the selected refill amount (STEP 510). The pharmaceutical injection supply manager can calculate a difference between the selected refill amount and an expected refill amount (STEP 512). The pharmaceutical injection supply manager can provide the difference via the interface (STEP 514).

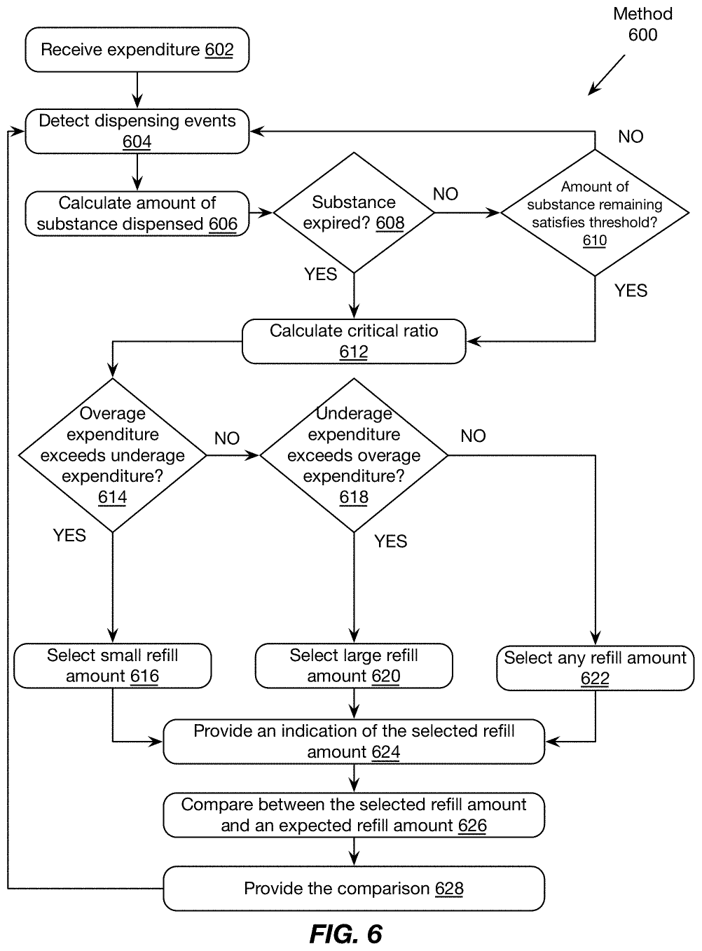

Now referring to FIG. 6, depicted is a flowchart diagram illustrating a method 600 for managing pharmaceutical injection supplies. The pharmaceutical injection supply manager 302 can perform the method 600. The pharmaceutical injection supply manager can receive expenditures via the interface (STEP 602). The pharmaceutical injection supply manager can detect dispensing events (STEP 604). The pharmaceutical injection supply manager can calculate an amount of substance dispensed (STEP 606). The pharmaceutical injection supply manager can identify whether the substance is expired (STEP 608). If the substance is not expired, the pharmaceutical injection supply manager can identify whether the amount of substance remaining satisfies a threshold (STEP 610). If the amount satisfies the threshold, the pharmaceutical injection supply manager can calculate the critical ratio (STEP 612). The pharmaceutical injection supply manager can identify whether an overage expenditure exceeds an underage expenditure (STEP 614). If the overage expenditure exceeds the underage expenditure, the pharmaceutical injection supply manager can select a small refill amount (STEP 616). If the overage expenditure does not exceed the underage expenditure, the pharmaceutical injection supply manager can identify whether the underage expenditure exceeds the overage expenditure (STEP 618). If the underage expenditure exceeds the overage expenditure, the pharmaceutical injection supply manager can select a large refill amount (STEP 620). If the underage expenditure does not exceed the overage expenditure, the pharmaceutical injection supply manager can select any refill amount (STEP 622). After selecting the refill amount, the pharmaceutical injection supply manager can provide an indication of the selected refill amount (STEP 624). The pharmaceutical injection supply manager can compare between the selected refill amount and an expected refill amount (STEP 626). The pharmaceutical injection supply manager can provide the comparison (STEP 628).

The pharmaceutical injection supply manager can receive expenditures (STEP 602). The pharmaceutical injection supply manager can receive expenditures via an interface. For example, the pharmaceutical injection supply manager can receive setup costs, substance costs, and substance usage to recommend the optimal amount of substance to load. The cost of a setup can include costs of the materials, such as cartridge, cannula, and site materials, which must be replaced every time there is a new site setup. The pharmaceutical injection supply manager can receive and store these costs. The cost of substance can be the cost of a unit of substance, which can be a standard unit of measure. The pharmaceutical injection supply manager can receive the cost of the unit of substance via the interfaces.

In some embodiments, the pharmaceutical injection supply manager can receive, from the interactive interface, a first expenditure per unit of the substance and a second expenditure per refill of the pump. The first expenditure can correspond to a cost per unit of the substance. For example, the first expenditure can identify a cost per unit of substance. The second expenditure can identify a cost to refill the pump with the substance. For example, the second expenditure can identify a cost to site prep (e.g. change caps, needles, pump locations, etc.) for the refill of the pump.

The pharmaceutical injection supply manager can detect dispensing events (STEP 604). Substance can be delivered by a pump (e.g., pump 214) to a user in a several ways. For example, the pharmaceutical injection supply manager can include data collection capabilities to automate the process of capturing each substance delivery (basal rate or bolus injection), sum, and transform the data as needed to calculate the refill amount. The pharmaceutical injection supply manager can receive expenditure information, such as substance cost, site prep cost and goal (optimized cost or target date/time). The pharmaceutical injection supply manager can identify, determine, or calculate a distribution shape, mean, and standard deviation of the dispensing events.

The pharmaceutical injection supply manager can detect, via a network receiver (e.g., network receiver 310) configured to receive dispensing activity of a pump (e.g., pump 214) communicatively coupled to the network receiver, dispensing events. Based on the dispensing events, the pharmaceutical injection supply manager can identify an amount of a substance that was dispensed or an expiration time of the substance. The pharmaceutical injection supply manager can identify or extract the timestamp, the amount, or the expiration time from each of the dispensing events. The pharmaceutical injection supply manager can identify predetermined refill amounts for the pump. For example, the pharmaceutical injection supply manager can identify that the pump can be refilled with 50 units of 100 units of substance.

The pharmaceutical injection supply manager can calculate an amount of substance dispensed (STEP 606). The pharmaceutical injection supply manager can identify or extract, from the plurality of dispensing events, an amount of substance dispensed. The pharmaceutical injection supply manager can identify a timestamp associated with each of the dispensing events. Based on the amount of substance dispensed and the timestamp, the pharmaceutical injection supply manager can calculate, identify, or extract an amount of substance dispensed per dispensing event. The pharmaceutical injection supply manager can identify the amount of substance dispensed per dispensing event as a basal rate and a bolus rate of substance dispensing. In some embodiments, the pharmaceutical injection supply manager can receive the amount of substance dispensed from the pump.

The pharmaceutical injection supply manager can identify whether the substance is expired (STEP 608). The pharmaceutical injection supply manager can receive an expiration time of the substance from the pump or via the interface. For example, the pharmaceutical injection supply manager can receive a date and/or time at which the substance in the pump cannot be dispensed. The pharmaceutical injection supply manager can store the expiration time. To identify or determine whether the substance is expired, the pharmaceutical injection supply manager can compare the expiration time to the current time. In some embodiments, the pharmaceutical injection supply manager can receive, from the pump, a status indicating whether the substance is expired. The pharmaceutical injection supply manager can store the status.

If the substance is not expired, the pharmaceutical injection supply manager can identify whether an amount of substance remaining satisfies a threshold (STEP 610). The pharmaceutical injection supply manager can identify the amount of substance remaining in the pump. By identifying the amount of substance that was dispensed the pharmaceutical injection supply manager can identify or calculate the amount of substance remaining in the pump. For example, if the pump was loaded with 100 units and the pharmaceutical injection supply manager detects five dispensing events of 10 units each, the pharmaceutical injection supply manager can calculate that the pump has 50 units of substance remaining.

The pharmaceutical injection supply manager can identify or receive the threshold corresponding to an amount of substance remaining in the pump at which the pump is to be refilled. For example, pharmaceutical injection supply manager can identify that the threshold is 50 units. The pharmaceutical injection supply manager can select or suggest to refill the pump when the amount of substance remaining satisfies (e.g., is less than) the threshold. Based on the previous example, the pharmaceutical injection supply manager can select or suggest refilling the pump when the amount of substance remaining is less than 50 units. The pharmaceutical injection supply manager can receive the threshold via the interface. The pharmaceutical injection supply manager can modify the threshold based on dispensing events. For example, the pharmaceutical injection supply manager can increase the threshold responsive to identifying that the pump frequently ran out of substance at the previous threshold. Conversely, the pharmaceutical injection supply manager can decrease the threshold responsive to identifying too many refills of the pump.

If the amount satisfies the threshold, the pharmaceutical injection supply manager can calculate a critical ratio (STEP 612). For example, if the amount of substance remaining is 45 units but the threshold is 50 units, the pharmaceutical injection supply manager can calculate or identify the critical ratio. The pharmaceutical injection supply manager can calculate or identify the critical ratio to identify a proportion between the expenditures and the amount of substance dispensed. The pharmaceutical injection supply manager can use the critical ratio to identify an optimal amount of substance to refill into the pump. The pharmaceutical injection supply manager can calculate or identify the critical ratio as

$$CR = \frac{{C}_{u}}{{C}_{u} + {C}_{o}} ,$$

which is between 0 and 1, inclusive. The critical ratio can be the cost of the underage divided by the sum of the cost of overage and cost of underage.

To calculate the critical ratio, the pharmaceutical injection supply manager can calculate or identify a mean and a standard deviation of the amount of the substance that was dispensed during the dispensing events. The pharmaceutical injection supply manager can calculate or identify the mean and the standard deviation from the each of the amounts of substance dispensed during the dispensing events. The pharmaceutical injection supply manager can use the mean to identify an average amount of substance dispensed per dispensing event.

Based on the mean and standard deviation, the pharmaceutical injection supply manager can generate the user's multi-day distribution from the dispensing events drawn from the pump. The pharmaceutical injection supply manager can identify a duration of the multi-day period based on the pump's substance capacity, the user's average dispensing usage, the maximum time between site changes, and any substance that is loaded but the pump cannot deliver. The period can be one, two, three, four, five, six, or more days. The daily usage can be determined from the pump's activities, as described above. The multi-day values are the sum of the daily values for the current and previous days covered by this period. The pharmaceutical injection supply manager can perform data transformation as needed, such as based on the best-fit distribution determined by the automated process.

Based on the calculated or identified mean and standard deviation, the pharmaceutical injection supply manager can calculate or identify a first metric based on a first expenditure. The first metric corresponds to the underage expenditure, which indicates the cost of discarding the substance that remains in the pump during the refill combined with the cost of refilling the pump. For example, the cost of underage can be the total of all costs associated with a new site prep, the cost of materials, any substance loaded into the cannula, and any loaded substance that the pump cannot deliver. The cost of the underage can be represented as Cᵤ. The pharmaceutical injection supply manager can calculate or identify a first metric by multiplying the first expenditure (e.g., per unit cost) by the amount of substance dispensed per dispensing event.

Based on the calculated or identified mean and standard deviation, the pharmaceutical injection supply manager can calculate or identify a second metric based on a second expenditure. The second metric corresponds to the overage expenditure, which indicates the per unit cost of the substance. The cost of the overage can be the per unit cost of substance multiplied by the user's average (mean) daily substance usage. The cost of the overage can be represented as Cₒ. The pharmaceutical injection supply manager can calculate or identify a second metric by adding the second expenditure (e.g., site prep cost) to a product of the first expenditure and an estimated amount of substance remaining in the pump at the expiration time of the substance. For example, if the refill amount is 500 units, the amount dispensed per dispensing event is 50, the pharmaceutical injection supply manager expects two dispensing events each day, and substance will expire in 4 days, the pharmaceutical injection supply manager can calculate that the amount of substance remaining at the expiration time will be 100 units.

In some embodiments, the pharmaceutical injection supply manager can calculate or identify the second metric by adding the second expenditure (e.g., site prep cost) to a product of the first expenditure and an estimated amount of substance remaining in the pump at an expected refill time of the substance. In some embodiments, the expected refill time is when the pump cannot dispense the necessary amount of substance. The pharmaceutical injection supply manager can identify or calculate the estimated amount of substance remaining based on a current amount of the substance and the amount of substance dispensed per dispensing event. For example, if the refill amount is 225 units and the amount dispensed per dispensing event is 50, the pharmaceutical injection supply manager can calculate that the amount of substance remaining at the refill time will be 25 units.

In some embodiments, the expected refill time is when the user of the pump prefers to refill the pump. The pharmaceutical injection supply manager can receive the expected refill time via the interface. For example, the expected refill time can be in the morning when the user prefers to refill the pump. For example, if the amount of substance in the pump is 500 units, the amount dispensed per dispensing event is 150, and the pharmaceutical injection supply manager expects two dispensing events each day, the pharmaceutical injection supply manager can calculate that the expected amount of substance remaining at the preferred refill time will be 200 units.

Since the amount of substance dispensed varies over time, the pharmaceutical injection supply manager can calculate a probability density function for the amount of substance dispensed. In some embodiments, based on the mean, standard deviation, and the amounts dispensed, the pharmaceutical injection supply manager can calculate or generate a probability density function for the dispensing events. Based on the probability density function, the pharmaceutical injection supply manager can manage or store probabilities for amounts of substance dispensed by the pump. The pharmaceutical injection supply manager can receive the amount dispensed as the input and output the probability of dispensing that amount. For example, if four dispensing events indicate that the pump dispensed 10 units, 15 units, 20 units, and 25 units, then the pharmaceutical injection supply manager can use the probability density function to identify that the probability of dispensing 10 units is 0.25.

The pharmaceutical injection supply manager can identify or calculate an inverse of the probability density function to identify the amount dispensed at a given probability. In some embodiments, the pharmaceutical injection supply manager can calculate the inverse based on the mean and the standard deviation. Based on the inverse, the pharmaceutical injection supply manager can transform the probabilities in the probability density function to the amount dispensed. To calculate the amount dispensed associated with the given probability, the pharmaceutical injection supply manager can receive the probability as the input and output the amount dispensed. For example, if the pump 214 dispenses 10 units, 15 units, 20 units, and 25 units, the pharmaceutical injection supply manager can receive a probability of 0.25 as an input and use the inverse to identify that the amount dispensed is 10 units, which indicates that there is a 25% chance of dispensing up to 10 units. Similarly, the pharmaceutical injection supply manager can receive a probability of 0.75 as an input and use the inverse to identify that the amount dispensed is 20 units, which includes the dispensing events of 10, 15, and 20 units and indicates that there is a 75% chance of dispensing up to 20 units.

The pharmaceutical injection supply manager can use the multi-day distribution, mean, standard deviation, and user preferences to calculate the amount of substance to be refilled. The pharmaceutical injection supply manager can calculate the inverse of the cumulative probability density function corresponding to the distributional shape identified at a given probability. The probability can be the probability of using all the substance prior to the end of the multi-day period for a given amount of loaded substance in the pump. The pharmaceutical injection supply manager can calculate the probability to be equal to the calculated critical ratio or the corresponding time preference from the user. In mathematical notation, the pharmaceutical injection supply manager can calculate F(Q) as the cumulative probability density function for a distributional shape with known mean and standard deviation, where Q is the quantity of substance loaded, and F⁻¹(⋅) can be the inverse of the function Q. Using the variables, the pharmaceutical injection supply manager can calculate the optimal amount to load as:

$${Q}^{*} = {F}^{- 1} ( C R ) = {F}^{- 1} ( \frac{{C}_{u}}{{C}_{u} + {C}_{o}} )$$

The pharmaceutical injection supply manager can provide the data into a model. The pharmaceutical injection supply manager can identify that the distribution of daily substance usage is lognormal. The pharmaceutical injection supply manager can be preconfigured with anonymous data of substance usage patterns. The pharmaceutical injection supply manager can perform a statistical ‘best fit’ test on several different distributions with ranges between zero and infinity. The pharmaceutical injection supply manager can identify a distributional shape for each user and include data that indicates the distributional shape, such as a lognormal distribution. The pharmaceutical injection supply manager can distinguish between a tradeoff in any prediction model between the amount of prior data to use and the relevancy of that data to the current use level. For example, the pharmaceutical injection supply manager can use a counterfactual analysis, multiple different lengths of prior data (between 3 and 90 days) for the recommended refill amount. The pharmaceutical injection supply manager can perform a test using, for example, a 7-day rolling average. The pharmaceutical injection supply manager can identify if the amount of substance loaded is different from the amount available for use in the pump. Therefore, the pharmaceutical injection supply manager can identify a measurement error. For example, the pharmaceutical injection supply manager can identify a measurement error of 0.4 mL, or 40 units of substance. The pharmaceutical injection supply manager can incorporate the measurement errors into the recommended load amount. For example, the pharmaceutical injection supply manager can add the measurement error to the recommended amount.

Based on the inverse of the probability density function and the critical ratio, the pharmaceutical injection supply manager can calculate or identify the refill amount. The refill amount can be an optimal amount of substance to load into the pump to balance between wasting substance and wasting resources for refilling. For example, the pharmaceutical injection supply manager can identify or calculate a distribution and fitting of data to a statistical distribution of values generated from the mean and standard deviation of dispensing usage.

The pharmaceutical injection supply manager can calculate a value of the site set up to either minimize costs and wasted substance or to give greater control of the duration of the process, based on user preference. For example, if less substance is loaded, then site changes will, on average, be more frequent, which would raise setup costs, but the amount of leftover substance will, on average, be lower, which would lower overage costs. The pharmaceutical injection supply manager can use usage data to identify a balance point between setup costs and overage costs. This pharmaceutical injection supply manager can use a model to balance overage and underage costs to provide a recommended refill amount.

The pharmaceutical injection supply manager can identify whether an overage expenditure exceeds an underage expenditure (STEP 614). The pharmaceutical injection supply manager can identify that the overage expenditure exceeds the underage expenditure if the critical ratio is between 0 and 0.5. For example, the formula for the critical ratio will approach 0 as the Cₒ increases relative to the Cᵤ. If the overage expenditure exceeds the underage expenditure, the pharmaceutical injection supply manager can select a small refill amount (STEP 616). By identifying that the overage expenditure exceeds the underage expenditure, the pharmaceutical injection supply manager can identify that the cost of refilling the pump is less the cost of the substance. When the cost of refilling is less than the cost of the substance, it would be beneficial to refill the pump often with small quantities to minimize wasting the more expensive substance during refills or due to expiration of the substance. Therefore, the pharmaceutical injection supply manager can select a small refill amount. As discussed in relation to STEP 612, since the amount of substance dispensed varies over time, the pharmaceutical injection supply manager can calculate the inverse of the probability density function to calculate the exact refill amount.

In some embodiments, the pharmaceutical injection supply manager can identify predetermined refill amounts of the substance. The pharmaceutical injection supply manager can receive the predetermined refill amounts from the pump, or the pharmaceutical injection supply manager can identify the predetermined refill amounts based on the dispensing events. For example, the pharmaceutical injection supply manager can identify frequent increases in the amount of substance remaining. For example, the pharmaceutical injection supply manager can identify frequent increases in 100, 200, or 300 units. The increases can correspond to the predetermined refill amounts. The predetermined refill amounts can correspond to a size of a cartridge with the substance. For example, the cartridges can be 100, 200, or 300 units.

The pharmaceutical injection supply manager can select the size of the cartridge based on the selected refill amount. For example, if the selected refill amount is 105 units and the cartridge sizes are 100 units and 200 units, the pharmaceutical injection supply manager can select the 100-unit cartridge.

If the overage expenditure does not exceed the underage expenditure, the pharmaceutical injection supply manager can identify whether the underage expenditure exceeds the overage expenditure (STEP 618). The pharmaceutical injection supply manager can identify that the underage expenditure exceeds the overage expenditure if the critical ratio is between 0.5 and 1. For example, the formula for the critical ratio will approach 1 as the Cᵤ increase relative to the Cₒ. If the underage expenditure exceeds the overage expenditure, the pharmaceutical injection supply manager can select a small refill amount (STEP 616). By identifying that the underage expenditure exceeds the overage expenditure, the pharmaceutical injection supply manager can identify that the cost of refilling the pump is more than the cost of the substance. When the cost of refilling is more than the cost of the substance, it would be beneficial to refill the pump often with large quantities. Even though refilling with large quantities may lead to more waste of the substance during refills or because of expiration, this approach can be less expensive because the cost of the refills exceeds the cost of the discarded substance. Therefore, the pharmaceutical injection supply manager can select a large refill amount. As discussed in relation to STEP 612, since the amount of substance dispensed varies over time, the pharmaceutical injection supply manager can calculate the inverse of the probability density function to calculate the exact refill amount.

If the underage expenditure does not exceed the overage expenditure and the overage expenditure does not exceed the underage expenditure, the pharmaceutical injection supply manager can select any refill amount (STEP 622). For example, the formula for the critical ratio will approach 0.5 if the Cᵤ equals the Cₒ. By identifying that the overage expenditure equals the underage expenditure, the pharmaceutical injection supply manager can identify that the cost of refilling the pump is equal to the cost of the substance. When the cost of refilling is equal to the cost of the substance, the user can refill the pump with any amount of substance. For example, the pharmaceutical injection supply manager can select a refill amount that correspond to the average (mean) amount of substance refilled.

After selecting the refill amount, the pharmaceutical injection supply manager can provide an indication of the selected refill amount (STEP 624). The pharmaceutical injection supply manager can provide the indication via the interactive interface. In some embodiments, the pharmaceutical injection supply manager can transmit the selected refill amount to a display of the pump.

The pharmaceutical injection supply manager can compare between refilling the selected refill amount and an expected refill amount (STEP 626). For example, the pharmaceutical injection supply manager can calculate the expected savings, in terms of dollars saved and reduction in wasted substance, by calculating the expected amount of money spent on substance waste if the user were to load substance based on their previous usage patterns. The pharmaceutical injection supply manager can provide the recommended load amounts via the client device along with the expected cost and substance savings that can be calculated from the usage patterns. The pharmaceutical injection supply manager can provide a comparison between loading the optimal amount and loading the average usage amount, a full cartridge, or a user's preference.

The pharmaceutical injection supply manager can identify a maximum refill amount, a mean refill amount (e.g., the amount the user typically refills), or a minimum refill amount for the value of the expected refill amount. The maximum refill amount can be the greatest amount of substance to refill into the pump. The expected refill amount can be the largest cartridge size of the substance. The minimum refill amount can be the least amount of substance to refill into the pump. The pharmaceutical injection supply manager can use the comparison to identify the benefits of refilling the selected amount instead of the expected amount (which can be associated with waste of the substance). To make the comparison between refilling the selected refill amount and the expected refill amount, the pharmaceutical injection supply manager can calculate a difference between the expected amount of substance remaining and the estimated amount of substance remaining.

To calculate the estimated amount of substance remaining responsive to selecting the selected refill amount, the pharmaceutical injection supply manager can calculate, identify, or retrieve the amount of substance dispensed per dispensing event. For example, if the selected refill amount is 300 units and the amount dispensed per dispensing event is 150, the pharmaceutical injection supply manager can calculate that the estimated amount of substance remaining is 0 units. In some embodiments, the pharmaceutical injection supply manager can calculate or identify the estimated amount of substance remaining based on an expected refill time. The pharmaceutical injection supply manager can receive the expected refill time via the interface. For example, the expected refill time can be in the morning when the user prefers to refill the pump. For example, if the selected refill amount is 300 units, the amount dispensed per dispensing event is 150, and the pharmaceutical injection supply manager expects two dispensing events each day, the pharmaceutical injection supply manager can calculate that the estimated amount of substance remaining at the preferred refill time will be 0 units.

The pharmaceutical injection supply manager can calculate or identify the expected amount of substance remaining based on the amount of substance dispensed per dispensing event. For example, if the expected refill amount is 500 units and the amount dispensed per dispensing event is 150, the pharmaceutical injection supply manager can calculate that the expected amount of substance remaining is 50 units (at which point the pump will not have enough substance for a dispensing event). In some embodiments, the pharmaceutical injection supply manager can calculate or identify the expected amount of substance remaining based on an expected refill time. The pharmaceutical injection supply manager can receive the expected refill time via the interface. For example, the expected refill time can be in the morning when the user prefers to refill the pump. For example, if the expected refill amount is 500 units, the amount dispensed per dispensing event is 150, and the pharmaceutical injection supply manager expects two dispensing events each day, the pharmaceutical injection supply manager can calculate that the expected amount of substance remaining at the preferred refill time will be 200 units.

The pharmaceutical injection supply manager can calculate or identify the difference between the expected amount of substance remaining and the estimated amount of substance remaining. In the aforementioned example, the pharmaceutical injection supply manager can calculate that the expected amount of substance remaining would be 200 units more than the estimated amount of substance remaining. The pharmaceutical injection supply manager can identify that the selected refill amount avoids discarding 200 units of the substance.

Based on the expected refill amount, the selected refill amount, and their respective amounts of substance remaining, the pharmaceutical injection supply manager can compare a frequency of refills. For example, if the expected refill amount is 500 units and the amount dispensed per dispensing event is 150, then the pharmaceutical injection supply manager can calculate that the frequency of refills would be one per three dispensing events. Likewise, if the selected refill amount is 300 units and the amount dispensed per dispensing event is 150, then the pharmaceutical injection supply manager can calculate that the frequency of refills would be one per two dispensing events. Based on the aforementioned examples, the pharmaceutical injection supply manager can calculate that the selected refill amount is associated with a 50% increase in the frequency of refills (despite the expected refill amount having an extra 67% of substance).

Based on the frequency of refills and the amount of substance expected to remain for discarding at each refill, the pharmaceutical injection supply manager can calculate or identify a cost of the expected refill amount and the selected refill amount. For example, the pharmaceutical injection supply manager can divide a summation of the cost of the refills and the cost of the discarded substance by the amount of substance refilled to calculate a cost per unit of substance. The cost of the discarded substance can be the first expenditure and the cost of the refills can be the second expenditure. The pharmaceutical injection supply manager can calculate the cost per unit of substance for the expected refill amount and the selected refill amount. In some embodiments, the expected refill amount and the selected refill amount can generate or identify an expenditure comparison based on (i) the first expenditure and the difference, and (ii) the second expenditure and a ratio between a first refill frequency of the pump based on the expected amount of substance and a second refill frequency of the pump based on the estimated amount of substance.

The pharmaceutical injection supply manager can provide the comparison (STEP 628). The comparison can include the difference between the selected refill amount and the expected refill amount, the minimum refill amount, or an average refill amount. In some embodiments, the pharmaceutical injection supply manager can provide the comparison via the interface. In some embodiments, the pharmaceutical injection supply manager can provide the difference to the display of the pump.

In some embodiments, the pharmaceutical injection supply manager can generate a graph that includes the amount of the substance that was dispensed during each of the plurality of dispensing events. The pharmaceutical injection supply manager can provide the difference to the interface. The pharmaceutical injection supply manager can calculate a line of best fit of the amount of the substance that was dispensed during each of the dispensing events. The pharmaceutical injection supply manager can display the graph with the line of best fit.

While the invention has been particularly shown and described with reference to specific embodiments, it should be understood by those skilled in the art that various changes in form and detail may be made therein without departing from the spirit and scope of the invention described in this disclosure.

While this specification contains many specific embodiment details, these should not be construed as limitations on the scope of any inventions or of what may be claimed, but rather as descriptions of features specific to particular embodiments of particular inventions. Certain features described in this specification in the context of separate embodiments can also be implemented in combination in a single embodiment. Conversely, various features described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms.

Thus, particular embodiments of the user matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

What is claimed:

1. A pharmaceutical injection supply manager, comprising:
a network receiver configured to receive dispensing activity of a pump communicatively coupled to the network receiver; and
one or more processors configured to:
detect, via the network receiver, a plurality of dispensing events, wherein each of the plurality of dispensing events includes a timestamp and an amount of a substance that was dispensed, the substance having one or more predetermined refill amounts and an expiration time,
calculate, from the plurality of dispensing events, an amount of substance dispensed per dispensing event,
select, based on the amount of substance dispensed per dispensing event and the expiration time, a refill amount of the one or more predetermined refill amounts, and
provide, via an interactive interface, an indication of the selected refill amount.

2. The pharmaceutical injection supply manager of claim 1, wherein the one or more processors are further configured to:
receive, from the interactive interface, a first expenditure per unit of the substance and a second expenditure per refill of the pump; and
select the refill amount based on the amount of substance dispensed per dispensing event, the expiration time, the first expenditure, and the second expenditure.

3. The pharmaceutical injection supply manager of claim 2, wherein the one or more processors are further configured to:
calculate a first metric by multiplying the first expenditure by the amount of substance dispensed per dispensing event;
calculate a second metric by adding the second expenditure to a product of the first expenditure and an estimated amount of substance remaining in the pump at the expiration time of the substance or an expected refill time of the substance, the estimated amount of substance remaining based on a current amount of the substance and the amount of substance dispensed per dispensing event; and
select the refill amount based on a proportion between the first metric and the second metric.

4. The pharmaceutical injection supply manager of claim 3, wherein the one or more processors are further configured to:
identify a probability density function of the plurality of dispensing events;
identify a mean and a standard deviation of the amount of the substance that was dispensed during the plurality of dispensing events; and
select the refill amount based on an inverse of the probability density function, the mean, the standard deviation, and the proportion.

5. The pharmaceutical injection supply manager of claim 2, wherein the one or more processors are further configured to:
identify, based on an expected refill amount of the one or more predetermined refill amounts and the amount of substance dispensed per dispensing event, an expected amount of substance remaining in the pump at a first expected refill time of the substance based on the expected refill amount;
identify, based on the selected refill amount and the amount of substance dispensed per dispensing event, an estimated amount of substance remaining in the pump at a second expected refill time of the substance based on the selected refill amount;
calculate a difference between the expected amount of substance remaining and the estimated amount of substance remaining; and
provide, via the interactive interface, the difference.

6. The pharmaceutical injection supply manager of claim 5, wherein the one or more processors are further configured to:
generate an expenditure comparison based on (i) the first expenditure and the difference, and (ii) the second expenditure and a ratio between a first refill frequency of the pump based on the expected amount of substance remaining and a second refill frequency of the pump based on the estimated amount of substance remaining; and
provide, via the interactive interface, the expenditure comparison.

7. The pharmaceutical injection supply manager of claim 1, wherein the one or more processors are further configured to:
receive, via the interactive interface, a preferred refill time of the substance; and
identify an estimated amount of substance remaining in the pump at the preferred refill time of the substance.

8. The pharmaceutical injection supply manager of claim 1, wherein the amount of substance dispensed per dispensing event comprises a basal rate and a bolus rate of substance dispensing.

9. The pharmaceutical injection supply manager of claim 1, wherein each of the one or more predetermined refill amounts of the substance corresponds to a size of a container containing the sub stance.

10. The pharmaceutical injection supply manager of claim 1, wherein the one or more processors are further configured to:
generate a graph comprising the amount of the substance that was dispensed during each of the plurality of dispensing events;
calculate a line of best fit of the amount of the substance that was dispensed during each of the plurality of dispensing events; and
display, via the interactive interface, the graph and the line of best fit.

11. A method for monitoring a pump supply, comprising:
detecting, by a computing device, via a network receiver configured to receive dispensing activity of a pump communicatively coupled to the network receiver, a plurality of dispensing events, wherein each of the plurality of dispensing events includes a timestamp and an amount of a substance that was dispensed, the substance having one or more predetermined refill amounts and an expiration time;
calculating, by the computing device, from the plurality of dispensing events, an amount of substance dispensed per dispensing event;
select, by the computing device, based on the amount of substance dispensed per dispensing event and the expiration time, a refill amount of the one or more predetermined refill amounts; and
providing, by the computing device, via an interactive interface, an indication of the selected refill amount.

12. The method of claim 11, wherein selecting the refill amount of the one or more predetermined refill amounts comprises:
receiving, by the computing device, from the interactive interface, a first expenditure per unit of the substance and a second expenditure per refill of the pump; and
selecting, by the computing device, the refill amount based on the amount of substance dispensed per dispensing event, the expiration time, the first expenditure, and the second expenditure.

13. The method of claim 12, further comprising:
calculating, by the computing device, a first metric by multiplying the first expenditure by the amount of substance dispensed per dispensing event;
calculating, by the computing device, a second metric by adding the second expenditure to a product of the first expenditure and an estimated amount of substance remaining in the pump at the expiration time of the substance or an expected refill time of the substance, the estimated amount of substance remaining based on a current amount of the substance and the amount of substance dispensed per dispensing event; and
selecting, by the computing device, the refill amount based on a proportion between the first metric and the second metric.

14. The method of claim 13, wherein selecting the refill amount of the one or more predetermined refill amounts comprises:
identifying, by the computing device, a probability density function of the plurality of dispensing events;
identifying, by the computing device, a mean and a standard deviation of the amount of the substance that was dispensed during the plurality of dispensing events; and
selecting, by the computing device, the refill amount based on an inverse of the probability density function, the mean, the standard deviation, and the proportion.

15. The method of claim 12, further comprising:
identifying, by the computing device, based on an expected refill amount of the one or more predetermined refill amounts and the amount of substance dispensed per dispensing event, an expected amount of substance remaining in the pump at a first expected refill time of the substance based on the expected refill amount;
identifying, by the computing device, based on the selected refill amount and the amount of substance dispensed per dispensing event, an estimated amount of substance remaining in the pump at a second expected refill time of the substance based on the selected refill amount;
calculating, by the computing device, a difference between the expected amount of substance remaining and the estimated amount of substance remaining; and
providing, by the computing device, via the interactive interface, the difference.

16. The method of claim 15, further comprising:
generating, by the computing device, an expenditure comparison based on (i) the first expenditure and the difference, and (ii) the second expenditure and a ratio between a first refill frequency of the pump based on the expected amount of substance and a second refill frequency of the pump based on the estimated amount of substance; and
providing, by the computing device, via the interactive interface, the expenditure comparison.

17. The method of claim 11, further comprising:
receiving, by the computing device, via the interactive interface, a preferred refill time of the substance; and
identifying, by the computing device, an estimated amount of substance remaining in the pump at the preferred refill time of the substance.

18. The method of claim 11, further comprising identifying, by the computing device, the amount of substance dispensed per dispensing event as a basal rate and a bolus rate of substance dispensing.

19. The method of claim 11, further comprising identifying, by the computing device, each of the one or more predetermined refill amounts of the substance as corresponding to a size of a cartridge with the substance.

20. The method of claim 11, further comprising:
generating, by the computing device, a graph comprising the amount of the substance that was dispensed during each of the plurality of dispensing events;
calculating, by the computing device, a line of best fit of the amount of the substance that was dispensed during each of the plurality of dispensing events; and
display, by the computing device, via the interactive interface, the graph having the line of best fit.
